(19) United States
(12) Patent Application Publication
(10) Pub. No.: US 2008/0107679 A1
(43) Pub. Date: May 8, 2008

(54) Skin care compositions including hexapeptide complexes and methods of their manufacture

(76) Inventors: Natalie Dilallo, Los Angeles, CA (US); Alyson Galderisi, Wayne, NJ (US)

Correspondence Address: Cislo & Thomas LLP, 1333 2nd Street, Suite #500, Santa Monica, CA, 90401-4110, US

(21) Appl. No.: 11/982,165

(22) Filed: Oct. 31, 2007

Related U.S. Application Data
(63) Continuation-in-part of application No. 11/347,587, filed on Feb. 4, 2006.
Application No. 11/347,587 is a continuation-in-part of application No. 10/918,135, filed on Aug. 13, 2004.
(60) Provisional application No. 60/495,574, filed on Aug. 14, 2003.

(51) Int. Cl. A61K 36/04

(52) U.S. Cl. 424/195.17

(57) ABSTRACT

Skin care compositions disclosed herein include, at a minimum, safe and effective amounts of at least one wrinkle reduction agent, which is a hexapeptide, and a natural exfoliating complex. The skin care compositions provide natural skin exfoliation, reduce fine lines and wrinkles, and improve skin elasticity and firmness.

### CROSS REFERENCE TO RELATED APPLICATIONS

This U.S. Continuation-in-Part patent Application claims the priority benefit of U.S. Continuation-in-Part application Ser. No. 11/347,587, filed Feb. 4, 2006, which claims priority of U.S. application Ser. No. 10/918,135, filed Aug. 13, 2004, which claims the benefit of U.S. Provisional Application Ser. No. 60/495,574, filed on Aug. 14, 2003, of which the entire contents of these applications are hereby incorporated by reference.

### BACKGROUND

1. Field of the Invention

The present invention relates to cosmetic compositions and, in particular, to multi-purpose skin care compositions.

2. Description of the Related Art

Various skin care products such as creams, lotions, bar soaps and gels are commercially available to cleanse, moisturize, exfoliate and minimize wrinkling of the skin. Typically, these skin care products are designed for a singular purpose. In order to achieve multiple benefits, an individual may have to use more than one skin care product. Recently, skin care products have been developed that may have more than one use such as a combination cleanser and moisturizer. However, there can be some difficulty in producing multi-use products as the physical properties of each individual component may not be compatible with one another. Also, these multi-purpose products may not be as effective when used alone. Accordingly, there remains a need for multi-purpose skin care products that can provide multiple skin care benefits.

### SUMMARY

Exemplary embodiments disclosed herein are directed to skin care compositions that include one or more ingredients that provide for natural skin exfoliation, reduce fine lines and wrinkles and improve skin elasticity and firmness. According to a first embodiment, the skin care composition is composed of a safe and effective amount of at least one anti-wrinkling agent and a safe and effective amount of a natural exfoliating complex.

In another embodiment, the anti-wrinkling agent is a hexapeptide. In another embodiment, the hexapeptide is an acetyl hexapeptide-3.

In yet another embodiment, the natural exfoliating complex is composed of ahnfeltia cocinna. In another exemplary embodiment the natural exfoliating complex also includes butylene glycol and glycosaminoglycans.

In yet another embodiment, the skin care compositions include a safe and effective amount of a hexapeptide or a hexapeptide complex, preferably in combination with one or more of the following ingredients: Willow Bark Extract, APT (glycosaminoglycans, water, butylenes glycol and Ahnfeltia extract), Moist 24(Imperate Cylindrica (Root) Extract, Water, Glycerin, PEG-8, and Carbomer), SK-Influx® (Ceramide 3, Ceramide 3 (B), Ceramide 6-II, Ceramide I, Cholesterol, Phytosphingosine, Carbomer, Xanthan Gum, Methylparaben, and Propylparaben), Polyolprepolymer-15, Dipalmitoyl Hydroxyproline (DPHP), Deliner (zea mays extract), and Oat Beta Glucan.

Other ingredients which may be combined with hexapeptide, or with hexapeptide and at least one ingredient selected from the group comprising Willow Bark Extract, APT (glycosaminoglycans, water, butylenes glycol and Ahnfeltia extract), Moist 24™ (Imperate Cylindrica (Root) Extract, Water, Glycerin, PEG-8, and Carbomer), SK-Influx® (Ceramide 3, Ceramide 3 (B), Ceramide 6-II, Ceramide I, Cholesterol, Phytosphingosine, Carbomer, Xanthan Gum, Methylparaben, and Propylparaben), Polyolprepolymer-15, Dipalmitoyl Hydroxyproline (DPHP), Deliner (zea mays extract), and Oat Beta Glucan, in accordance with the present invention include: other peptides including Oligopeptide, pentapeptide, pentapeptide-3, hexapeptide-10, tripeptide, palmitoyl tripeptide-3, tripeptide-1, palmitoyl peptide-3, palmitoyl oligopeptide, and acetyl teterapeptdie-5, hydrolyzed wheat, soy, and rice protein, soybean protein, white tea, green tea, Tocopheryl, Retinyl palmitate, ascrobyl palmitate, olive oil, fruit extract including lemon, orange, grapefruit, melon, grape, fennel, shea butter, cucumber, and banana, phospholipids, grape seed extract, licorice extract, cola nitida seed extract, Paullina cupana seed extract, ilex paraguariensis, hops extract Melissa officinalis leaf extract, mistletoe extract, chamomilla reticutita (matricaria) flower extract, achillea milleforlium extract, allantoin, chrysin, aloe barbadensis leaf juice, dimethylmethoxy chromol, saccharide isomerate, oxido reductases, cotton, pantothenic acid, methyldihydrojasmonate, meridamate, isostearyl palmitate, lecithin, magnesium ascorbyl phosphate, tricaprin, citrus fruit peel including grapefruit peel, citrus aurantium, cacao seed butter, iris florentina root extract, hypnea musciformis extract, gellidiela acerosa extract, sargassum filipendula extract, rosmarinus officinalis, equisetum arvense extract, pine cone extract, wheat germ extract, soybean germ extract, jojoba wax, oxidized polyethylene homopolymer, synthetic iron oxide, dimethylmethoxy chromanol, algae extract, pullulan, ascorbic acid, glucoside, and combinations thereof.

In yet another embodiment, the compositions contain at least three ingredients selected from the following group: a hexapeptide complex, Willow Bark Extract, APT, Moist 24, SK-Influx™, Polyolprepolymer-15, DPHP, Deliner, and Oat Beta Glucan.

In other embodiments, the skin care composition may include one or more additional components such as, but not limited to, conditioning agents, skin protectants, antioxidants, sunscreen actives, cleansing agents, cosmetic soothing aids, or the like.

### DETAILED DESCRIPTION

The detailed description set forth below is intended as a description of exemplary embodiments and is not intended to represent the only forms in which the exemplary embodiments may be constructed and/or utilized. The description sets forth the functions and the sequence of steps for constructing and/or operating the exemplary embodiments. However, it is to be understood that the same or equivalent functions and sequences may be accomplished by different exemplary methods are also intended to be encompassed within the spirit and scope of the invention.

As used herein, “safe and effective amount” means a sufficient amount of a compound, composition or other material described by this phrase to significantly induce a positive modification in the condition being treated, but low enough to avoid undue side effects (e.g., significant skin irritation or sensitization), within the scope of sound judgment of the skilled person. The safe and effective amount of the compound, composition or other material may vary with the particular skin being treated, the age and physical condition of the biological subject being treated, the severity of the condition, the duration of treatment, the nature of concurrent therapy, the specific compound, composition, or other material employed, the particular cosmetically acceptable topical carrier utilized, and the factors within the knowledge and expertise of the skilled person.

Exemplary embodiments disclosed herein are directed to skin care compositions that include one or more ingredients that provide a plurality of skin care benefits such as, but not limited to, exfoliating the skin naturally, reducing fine lines and wrinkles and improving skin elasticity and firmness.

According to a first embodiment, the skin care composition is composed of a safe and effective amount of at least one anti-wrinkling agent and a safe and effective amount of a natural exfoliating complex.

In another embodiment, the anti-wrinkling agent is a hexapeptide. In another embodiment, the hexapeptide is an acetyl hexapeptide-3.

In yet another embodiment, the natural exfoliating complex is composed of ahnfeltia cocinna. In another exemplary embodiment the natural exfoliating complex also includes butylene glycol and glycosaminoglycans.

In yet another embodiment, the skin care compositions include a safe and effective amount of a hexapeptide or a hexapeptide complex, preferably in combination with one or more of the following ingredients: Willow Bark Extract, APT™ (glycosaminoglycans, water, butylene glycol and Ahnfeltia extract), Moist 24™ (Imperate Cylindrica (Root) Extract, Water, Glycerin, PEG-8, and Carbomer), Sk-Influx™ (Cera, Polyolprepolymer-15, Dipalmitoyl Hydroxyproline (DPHP), Deliner (zea mays extract), and Oat Beta Glucan.

Other ingredients which may be combined with hexapeptide, or with hexapeptide and at least one ingredient selected from the group comprising Willow Bark Extract, APT (glycosaminoglycans, water, butylenes glycol and Ahnfeltia extract), Moist 24™ (Imperate Cylindrica (Root) Extract, Water, Glycerin, PEG-8, and Carbomer), SK-Influx® (Ceramide 3, Ceramide 3 (B), Ceramide 6-II, Ceramide I, Cholesterol, Phytosphingosine, Carbomer, Xanthan Gum, Methylparaben, and Propylparaben), Polyolprepolymer-15, Dipalmitoyl Hydroxyproline (DPHP), Deliner (zea mays extract), and Oat Beta Glucan, in accordance with the present invention include: other peptides including Oligopeptide, pentapeptide, pentapeptide-3, hexapeptide-10, tripeptide, palmitoyl tripeptide-3, tripeptide-1, palmitoyl peptide-3, palmitoyl oligopeptide, and acetyl teterapeptdie-5, hydrolyzed wheat, soy, and rice protein, soybean protein, white tea, green tea, Tocopheryl, Retinyl palmitate, ascrobyl palmitate, olive oil, fruit extract including lemon, orange, grapefruit, melon, grape, fennel, shea butter, cucumber, and banana, phospholipids, grape seed extract, licorice extract, cola nitida seed extract, Paullina cupana seed extract, ilex paraguariensis, hops extract Melissa officinalis leaf extract, mistletoe extract, chamomilla reticutita (matricaria) flower extract, achillea milleforlium extract, allantoin, chrysin, aloe barbadensis leaf juice, dimethylmethoxy chromol, saccharide isomerate, oxido reductases, cotton, pantothenic acid, methyldihydrojasmonate, meridamate, isostearyl palmitate, lecithin, magnesium ascorbyl phosphate, tricaprin, citrus fruit peel including grapefruit peel, citrus aurantium, cacao seed butter, iris florentina root extract, hypnea musciformis extract, gellidiela acerosa extract, sargassum filipendula extract, rosmarinus officinalis, equisetum arvense extract, pine cone extract, wheat germ extact, soybean germ extract, jojoba wax, oxidized polyethylene homopolymer, synthetic iron oxide, dimethylmethoxy chromanol, algae extract, pullulan, ascorbic acid, and glucoside.

In yet another embodiment, the compositions contain at least three ingredients selected from the following group: a hexapeptide complex, Willow Bark Extract, APT, Moist 24, Sk-Influx™, Polyolprepolymer-15, Dipalmitoyl Hydroxyproline (DPHP), Deliner (zea mays extract), and Oat Beta Glucan.

In other embodiments, the skin care composition may include one or more additional components such as, but not limited to, conditioning agents, skin protectants, antioxidants, sunscreen actives, cleansing agents, cosmetic soothing aids, or the like.

The hexapeptide complex is an anti aging ingredient that is chemically combined from naturally derived amino acids that minimizes and softens fine lines. It has been found to be a highly effective wrinkle reducer of the fine to medium depth “expression” lines, and specifically targets the repeated, biomechanical, muscular contractions of facial expressions such as laughing, squinting, frowning, etc. by functioning to reduce the intensity of these muscle contractions that overtime engrave wrinkles in the skin. It also helps to relax muscles, as opposed to paralyzing them, like Botox™, thereby reducing the appearance of wrinkles without toxicity and without the loss of natural expression. Thus, it offers an injection free, non-toxic alternative to Botox™. The preferred hexapeptide is acetyl hexapeptide-3, sold under the name Argireline™. Depending on the application of the skin care composition, the amount of hexapeptide and other ingredients used may be varied. The skin care composition may include hexapeptide complexes having hexapeptide concentrations ranging from approximately 0.00001 to approximately 15% w/w, preferably from 0.01% w/w to 10% w/w, and more preferably from approximately 0.1 to 5%.

The hexapeptide component of the hexapeptide complex has been clinically shown to minimize wrinkling of the skin. As those skilled in the art will appreciate, wrinkling of the skin can be attributed to natural aging and overexposure to the sun. Although the mechanism of wrinkle formation is not completely known, studies have shown that visible fine wrinkles may be attributable to a reduction in the number and size of elastic fibers in the papillary dermis, an atrophy of the dermis and a reduction in subcutaneous adipose tissue. With respect to coarse wrinkles, studies have shown that these wrinkles may be attributed to excessive deposition of abnormal elastic elements in the upper dermis and thickening of the skin.

The hexapeptide component has been shown to reduce wrinkle formation by reducing the release of catecholamines and limiting the production of SNARE complexes, which are membrane proteins that regulate neurotransmitter release. Studies have shown that minimizing the production of the SNARE complexes decreases the formation of coarse wrinkles. Additionally, the hexapeptide compound has been shown to minimize the overproduction and release of catecholamines, which are attributable to wrinkle formation and fine wrinkles. APT is a natural, mild, exfoliation medium that increases cellular turnover (fibroblast proliferation). This component has been shown to increase turnover by approximately 18% within about 4 weeks, without the over stimulation commonly caused by the use of alpha hydroxyacids or retinal that commonly irritated and cause hyperplasia (an uneven thickening of the skin in an erratic formation). Thus, cells rebuild more slowly and in a more uniform and organized manner like a brick and mortar fashion. This slow rebuilding process ensures a more perfected, symmetrical, cell alignment, and therefore, a more uniform skin health, firmness, elasticity, and tonality or skin opacity. Raw material cell culture studies show that the newly rebuilt cells possess a high water binding capacity that provide intense, immediate, super hydration. Also, the APT can increase the density of the collagen bundles of the upper dermis, which can lead to greater skin elasticity. The Ahnfeltia Concinna component of APT provides for the natural exfoliation of skin. In contrast to prior art exfoliants such as AHA, APT GC can permit the uniform exfoliation of the skin. That is, while AHA can increase skin cell turnover (i.e. exfoliate the top layer(s) of skin), it is particularly harsh and may cause “skin shock” that may cause redness and irritation of the skin. Accordingly, the underlying skin layers may regenerate at different rates leading to uneven skin appearance. Furthermore, AHA can have different rates of penetrating the skin that may also contribute to blotchiness that can be associated with exfoliation procedures.

In contrast to prior art exfoliants, APT can improve skin tone by approximately 15% after 4 weeks of use. The APT component can be more uniformly absorbed by the skin to reduce blotchiness. Also, pore size can be reduced by approximately 35% and skin texture can be improved by approximately 42% after 4 weeks of use. Furthermore, because the APT component does not utilize harsh chemicals, redness and irritation can be reduced. With respect to skin redness, the skin care composition can improve skin redness (i.e., lessen skin redness) by approximately 25% after 2 weeks and by approximately 33% after 4 weeks of use. With respect to skin irritation, the skin care composition can decrease skin irritation by approximately 9% after 2 weeks and approximately 23% after 4 weeks of use. The reduction of redness and irritation is believed to be due to the APT and willow bark extract. The concentration of APT contained in the formulations preferably ranges from 0 to approximately 5% w/w, more preferably from approximately 0.01% w/w to approximately 2% w/w, and more preferably at about 2%.

The willow bark extract is a natural exfoliating extract that can increase the cellular renewal capability of the formulations better than salicylic acid and with less irritation. The willow bark extract also creates a general improvement in the skin's appearance that results in smother skin and a reduction in fine lines and wrinkles. The willow bark extract that is used in the compositions of the present invention is preferably white willow bark which has an irritation factor comparable to that of glycerin tat registers at an almost non-existent level. It is therefore a well suited exfoliation vehicle for aging, dry, dehydrated, and sensitive skin. The amount of willow bark extract used in the formulations of the present invention ranges from approximately 0 to 5% w/w, and more preferably from between 0.2 and 2.5%. According to other embodiments, the efficacious amount is approximately 1% and higher.

The Moist 24 component is a plant based, twenty-four hour, long lasting moisturizer that is beneficial for the continuous hydration of aging skin. It is also excellent for dry, dehydrated skin that is deficient in potassium, since potassium balances the acid/alkaline system in the body and works with sodium to regulate the skin's water balance among many other functions. The Moist 24 used in the formulations of the present invention preferably ranges from between approximately 0 and 3% w/w, more preferably between approximately 0 and 1%, and is more preferably around 0.25%.

DPHP reduces wrinkles caused by physiological aging. It defends against both natural and photo-induced skin aging factors, including weakening of the collagen fiber network, degrading of the dermal matrix and the dermal/epidermal junction, and free radical damage that has mediated to skin cells within the fibroblasts. It further helps to prevent resulting skin aging effects including flaccid, lose skin, loss of tone and firmness, development of wrinkles and crow's feet, skin structural loss and moisture content. Additionally, it delivers a complex, triple action effect upon aging including anti-wrinkle, effects, long lasting lipophilic moisturizing activity, and skin firming by stimulating pro-collagen production to protect the extra cellular dermal matrix, protecting the elastin in the skin, and protecting fibroblasts against free radicals. The preferred amount of DPHP used in the formulations of the present invention ranges between 0 and approximately 1% w/w.

The SK-Influx component effectively enhances skin moisture because of its unique skin identical lipid concentration. This ingredient consists of a multi-lamellar (membrane) system closely mimicking the structure and function of the lipid barrier in the stratum corneum, and contains a range of ceramides, free phytosphingosine and cholesterol thereby replenishing the skin's various lipid classes. It improves skin barrier function and therefore provides exceptional protection for the skin. It further enhances the delivery and exchange of skin lipids to facilitated “lipid dry” skin conditions as opposed to “water dry” conditions, and is ideal for aging, dry, dehydrated and sensitive skin that in most cases, commonly experiences depleted lipid content. The preferred amount of SK-Influx used in the compositions of the present invention ranges from 0 to approximately 5%, and more preferably from between approximately 3-5% w/w.

Polyolprepolymer-15 is a component which improves the deposition of actives in the upper layers of the skin and creates a well suited delivery systems for the active ingredients in the compositions of the present invention. It ensures a slow, but optimum timing of the deposition of the active components due to its chemical structure and molecular weight, creating within the stratum cornium a long lasting liquid reservoir for release of various materials in the skin. The prepolyolprepolymer-15 component is particularly ideal for compositions which have a high amount of active ingredients, since it slows their penetration in a timed release manner that does not shock the skin. The amount of polyolprepolymer-15 is approximately 2%.

As the skin ages, the prefect integrity of the extra-cellular matrix, which is a combination of collagen, elastin, and glycosaminoglycans, undergoes alterations causing wrinkles to appear. The Deliner component stimulates fibronectin synthesis, which is the webbing of the extra-cellular matrix, which in turn, enables fibroblast to migrate to those areas of the matrix that have been altered or badly damaged. As a result, fibroblastic synthesis is reactivated and reforms the extra-cellular matrix. Ultimately the repaired matrix provides support and firmness to the skin tissue causing wrinkles to fade. The preferred amount of Deliner used in the compositions of the present invention may range from 0 to approximately 2% w/w, and is more preferably around 1%.

Oat Beta Glucan is a natural, soothing plant derivative which has anti-inflammatory properties. This component increases collagen synthesis and cell renewal, reduces fine lines and wrinkles, protects the skin from UV damage, and serves as a protective film-forming agent. The preferred amount of Oat Beta Glucan used in the compositions of the present invention may range from approximately 0 to 5% w/w, and more preferably between 1 and 2%.

The combination of ingredients of the skin care compositions of the present invention are believed to provide synergistic benefits which are otherwise not available with any one of the components used by itself. Clinical evaluations based on the use of the hexapeptide complex itself evidenced a reduction in fine lines and wrinkles, including laugh lines, frown lines, and smile lines, by approximately 13% after 2 weeks and approximately 33% in 4 weeks of use. It was further observed that 100% of participants experienced a reduction in under-eye fine lines and wrinkles on the average of approximately 16% after 4 weeks and approximately 52% after 8 weeks of use. Additionally, an increase in moisture content by 37% after 15 minutes of use was observed.

It is further believed that the compositions of the present invention may improve skin complexion causing the skin to be more flawless and even-toned. Additionally, it is believed that the use of the compositions may increase cellular turnover by 100% with minimal irritation after approximately 2 to 4 weeks of use.

Furthermore, in other exemplary embodiments, the skin care composition may include other components including, but not limited to, conditioning agents, skin protectants, antioxidants, UV absorbing agents, sunscreen actives, cleansing agents, viscosity modifying agents, film formers, emollients, surfactants, solubilizing agents, preservatives, fragrance, chelating agents, foaming or antifoaming agents, opacifying agents, stabilizing agents, pH adjustors, absorbents, anti-caking agents, slip modifiers, various solvents, solubilizing agents, denaturants, abrasives, bulking agents, emulsion stabilizing agents, suspending agents, colorants, binders, conditioning agent-emollients, surfactant emulsifying agents, biological products, anti-acne actives, anti-wrinkle and anti-skin atrophy actives, skin barrier repair aids, cosmetic soothing aids, topical anesthetics, artificial tanning agents and accelerators, skin lightening actives, antimicrobial and antifungal actives, sebum stimulators, sebum inhibitors, humectants, and/or combinations thereof.

Conditioning agents may generally be used to improve the appearance and/or feel of the skin upon and after topical application via moisturization, hydration, plasticization, lubrication, and occlusion, or a combination thereof. Non-limiting examples of suitable conditioning agents are described in the CTFA Cosmetic Ingredient Handbook, Second Edition, 1992, which is incorporated by reference herein in its entirety.

According to one exemplary embodiment, the conditioning component may be composed of a water-soluble conditioning agent, an oil soluble conditioning agent, a conditioning emulsion, or any combination or permutation thereof.

Non-limiting examples of oil soluble conditioning agents include, but are not limited to, mineral oil (see The Merck Index, Tenth Edition, Entry 7048, p. 1033 (1983) and International Cosmetic Ingredient Dictionary, Fifth Edition, vol. 1, p. 415-417 (1993)), petrolatum (see The Merck Index, Tenth Edition, Entry 7047, p. 1033 (1983); Schindler, Drug. Cosmet. Ind., 89, 36-37, 76, 78-80, 82 (1961), and International Cosmetic Ingredient Dictionary, Fifth Edition, vol. 1, p. 537 (1993)), C7-C40 branched chain hydrocarbons, C1-C30 alcohol esters of C1-C30 carboxylic acids, C1-C30 alcohol esters of C2-C30 dicarboxylic acids, monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, propylene glycol diesters of C1-C30 carboxylic acids, C1-C30 carboxylic acid monoesters and polyesters of sugars, polydialkylsiloxanes, polydiarylsiloxanes, polyalkarylsiloxanes, cylcomethicones having 3 to 9 silicon atoms, vegetable oils, hydrogenated vegetable oils, polypropylene glycol C4-C20 alkyl ethers, di C8-C30 alkyl ethers, and mixtures thereof.

Non-limiting examples of straight and branched chain hydrocarbons having from about 7 to about 40 carbon atoms include, but are not limited to, dodecane, isododecane, squalane, cholesterol, hydrogenated polyisobutylene, docosane (i.e. a C22 hydrocarbon), hexadecane, isohexadecane (a commercially available hydrocarbon sold as Permethyl® 101A by Presperse, South Plainfield, N.J.). Also useful are the C7-C40 isoparaffins, which are C7-C40 branched hydrocarbons.

Also useful are C1-C30 alcohol esters of C1-C30 carboxylic acids and of C2-C30 dicarboxylic acids, including straight and branched chain materials as well as aromatic derivatives, esters such as monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, and propylene glycol diesters of C1-C30 carboxylic acids, including straight chain, branched chain and aryl carboxylic acids, and propoxylated and ethoxylated derivatives of these materials. Examples of these include diisopropyl sebacate, diisopropyl adipate, isopropyl myristate, isopropyl palmitate, myristyl propionate, ethylene glycol distearate, 2-ethylhexyl palmitate, isodecyl neopentanoate, di-2-ethylhexyl maleate, cetyl palmitate, myristyl myristate, stearyl stearate, cetyl stearate, behenyl behenrate, dioctyl maleate, dioctyl sebacate, diisopropyl adipate, cetyl octanoate, diisopropyl dilinoleate, caprilic/capric triglyceride, PEG-6 caprylic/capric triglyceride, PEG-8 caprylic/capric triglyceride, cetyl ricinoleate, cholesterol hydroxystearate, cholesterol isostearate, and mixtures thereof.

Also useful are various C1-C30 monoesters and polyesters of glycerin and related materials, which are derived from glycerin and one or more carboxylic acid moieties. Depending on the constituent acid and glycerin, these esters can be in either liquid or solid form at room temperature. Non-limiting examples of solid esters include glyceryl tribehenate, glyceryl stearate, glyceryl palmitate, glyceryl distearate, and glyceryl dipalmitate.

Also useful are various C1-C30 monoesters and polyesters of sugars and related materials. Depending on the constituent acid and sugar, these esters can be in either liquid or solid form at room temperature. Examples of liquid esters include glucose tetraoleate, the glucose tetraesters of soybean oil fatty acids (unsaturated), the mannose tetraesters of mixed soybean oil fatty acids, the galactose tetraesters of oleic acid, the arabinose tetraesters of linoleic acid, xylose tetralinoleate, galactose pentaoleate, sorbitol tetraoleate, the sorbitol hexaesters of unsaturated soybean oil fatty acids, xylitol pentaoleate, sucrose tetraoleate, sucrose pentaoletate, sucrose hexaoleate, sucrose hepatoleate, sucrose octaoleate, and mixtures thereof. Examples of solid esters include: sorbitol hexaester in which the carboxylic acid ester moieties are palmitoleate and arachidate in a 1:2 molar ratio; the octaester of raffinose in which the carboxylic acid ester moieties are linoleate and behenate in a 1:3 molar ratio; the heptaester of maltose wherein the esterifying carboxylic acid moieties are sunflower seed oil fatty acids and lignocerate in a 3:4 molar ratio; the octaester of sucrose wherein the esterifying carboxylic acid moieties are oleate and behenate in a 2:6 molar ratio; and the octaester of sucrose wherein the esterifying carboxylic acid moieties are laurate, linoleate and behenate in a 1:3:4 molar ratio. An exemplary solid material is sucrose polyester in which the degree of esterification is 7-8, and in which the fatty acid moieties are C18 mono- and/or di-unsaturated and behenic, in a molar ratio of unsaturates:behenic of 1:7 to 3:5. Another exemplary solid sugar polyester is the octaester of sucrose in which there are about 7 behenic fatty acid moieties and about 1 oleic acid moiety in the molecule. Other materials include cottonseed oil or soybean oil fatty acid esters of sucrose.

Nonvolatile silicones such as polydialkylsiloxanes, polydiarylsiloxanes, and polyalkarylsiloxanes are also useful oils. The polyalkylsiloxanes correspond to the general chemical formula R3 SiO[R₂ SiO]ₓ SiR₃ wherein R is an alkyl group (preferably R is methyl or ethyl, more preferably methyl) and x is an integer up to about 500, chosen to achieve the desired molecular weight. Commercially available polyalkylsiloxanes include the polydimethylsiloxanes, which are also known as dimethicones, non-limiting examples of which include the VICASIL® series sold by General Electric Company and the DOW CORNING® 200 series sold by Dow Corning Corporation. Specific examples of polydimethylsiloxanes useful herein include DOW CORNING® 225 fluid having a viscosity of 10 centistokes and a boiling point greater than 200° C., and DOW CORNING® 200 fluids having viscosities of 50, 350, and 12,500 centistokes, respectively, and boiling points greater than 200° C. Also useful are materials such as trimethylsiloxysilicate, which is a polymeric material corresponding to the general chemical formula [(CH₂)₃ SiO_{1/2}]ₓ [SiO₂]_{y}, wherein x is an integer from about 1 to about 500 and integer from about 1 to about 500. A commercially available trimethylsiloxysilicate is sold as a mixture with dimethicone as DOW CORNING® 593 fluid. Also useful herein are dimethiconols, which are hydroxy terminated dimethyl silicones. These materials can be represented by the general chemical formulas R₃ SiO[R₂SiO]ₓ SiR₂ OH and HOR₂ SiO[R₂ SiO]ₓ SiR₂ OH wherein R is an alkyl group (preferably R is methyl or ethyl, more preferably methyl) and x is an integer up to about 500, chosen to achieve the desired molecular weight. Commercially available dimethiconols are typically sold as mixtures with dimethicone or cyclomethicone (e.g. DOW CORNING® 1401, 1402, and 1403 fluids). Also useful herein are polyalkylaryl siloxanes, with polymethylphenyl siloxanes having viscosities from about 15 to about 65 centistokes at 25° C. being preferred. These materials are available, for example, as SF 1075 METHYLPHENYL FLUID (sold by General Electric Company) and 556 COSMETIC GRADE PHENYL TRIMETHICONE FLUID (sold by Dow Corning Corporation).

Vegetable oils and hydrogenated vegetable oils are also useful herein. Examples of vegetable oils and hydrogenated vegetable oils include safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, hydrogenated safflower oil, hydrogenated castor oil, hydrogenated coconut oil, hydrogenated cottonseed oil, hydrogenated menhaden oil, hydrogenated palm kernel oil, hydrogenated palm oil, hydrogenated peanut oil, hydrogenated soybean oil, hydrogenated rapeseed oil, hydrogenated linseed oil, hydrogenated rice bran oil, hydrogenated sesame oil, hydrogenated sunflower seed oil, and mixtures thereof.

Also useful are C4-C20 alkyl ethers of polypropylene glycols, C1-C20 carboxylic acid esters of polypropylene glycols, and di-C8-C30 alkyl ethers. Non-limiting examples of these materials include PPG-14 butyl ether, PPG-15 stearyl ether, dioctyl ether, dodecyl octyl ether, and mixtures thereof.

Other useful oil soluble conditioning agents include CREMEROL (available from Amerchol), ELDEW CL301 (available from Ajinomoto), MODULAN (an acetylated lanolin which is commercially available from Croda), OHLAN (a hydroxylated lanolin which is commercially available from Amerchol), phytantriol, super sterol esters, such as C₁-C₃₀ cholesteroltlanosterol esters, (available from Croda), and mixtures thereof.

Non-limiting examples of conditioning agents useful as water soluble conditioning agents include those selected from the group consisting of polyhydric alcohols, polypropylene glycols, polyethylene glycols, ureas, pyrolidone carboxylic acids, ethoxylated and/or propoxylated C3-C6 diols and triols, alpha-hydroxy C2-C6 carboxylic acids, ethoxylated and/or propoxylated sugars, polyacrylic acid copolymers, sugars having up to about 12 carbons atoms, sugar alcohols having up to about 12 carbon atoms, and mixtures thereof. Specific examples of useful water soluble conditioning agents include materials such as urea; guanidine; glycolic acid and glycolate salts (e.g. ammonium and quaternary alkyl ammonium); lactic acid and lactate salts (e.g. ammonium and quaternary alkyl ammonium); sucrose, fructose, glucose, eruthrose, erythritol, sorbitol, mannitol, glycerol, hexanetriol, propylene glycol, butylene glycol, hexylene glycol, and the like; polyethylene glycols such as PEG-2, PEG-3, PEG-4, PEG-5, PEG-6, PEG-8, PEG-9, PEG-10, PEG-15 PEG-30, PEG-50, polypropylene glycols such as PPG-9, PPG-12, PPG-15, PPG-17, PPG-20, PPG-26, PPG-30, PPG-34; alkoxylated glucose; hyaluronic acid; and mixtures thereof. Also useful are materials such as aloe vera in any of its variety of forms (e.g., aloe vera gel), chitin, starch-grafted sodium polyacrylates such as SANWET® IM-1000, IM-1500, and IM-2500 (available from Celanese Superabsorbent Materials, Portsmouth, Va.); lactamide monoethanolamine; acetamide monoethanolamine; and mixtures thereof. Also useful are propoxylated glycerols. Other useful water soluble conditioning agents include arginine, arginine asparate (available from Ajinomoto), ARGININE PCA (available from Argidone-UCIB), 1,3 butylene glycol, CHITOLAM NB/101 (available from Lamberti), chitosan salts, Codiavelane (available from Secma), COLLAGEN AMINO ACID (available from Crotein CAA-Croda), creatine, dextran, dextrin, diglycerol, dipropylane glycol, ectoines, erythritol, FUCOGEL (available from Solabia), fructose, glucamine salts, glucose glutamate (commercially available as WICKENOL 545 from Caschem), glucuronic acid salts, glutamic acid salts, glycereth 12, glycereth 20, glycereth 7, glycerin, glyceryl PCA, glycogen, hexylene glycol, honey, hyaluronic acid, hydrogenated honey, hydrogenated starch hydrolysates, hydrolyzed mucopolysaccharides, hydroxy proline, Indinyl CA (available from Laboratoires Serobiologiques), inositol, keratin amino acids (commercially available as CROTEIN HKP from Croda), konjac mannan, Larex A-200 (available from Larex), LYSINE PLA (commercially available as LYSIDONE from UCIB), maltitol, maltose, mannitol, mannose, Mariscan (available from Pentapharm), Melhydrin (available from laboratories Serobiologiques), methoxy PEG 10, methoxy, methyl gluceth 10 (commercially available as GLUCAM E10 from Amerchol), methyl gluceth 20 (commercially available as GLUCAM E20 from Amerchol), methyl glucose, 3 methyl 1,3 butandiol, N acetyl glucosamine salts, panthenol, PEG 15 butanediol, butanediol, PEG 5 pentaerythitol, pentaerythitol, Pentaglycan (available from Pentapharm), 1,2 pentanediol, phytohyaluron (jute extract), polyglycerol sorbitol, PPG 1 glyceryl ether, proline, propylene glycol, 2 pyrrolidone-5-carboxylic acid and its salts, saccharide isomerate (commercially available as PENTAVITIN from Pentapharm), Seacare (available from Secma), Sericin (available from Pentapharm), serine, silk amino acids (commercially available as CROSLIK LIQUID from Croda), sodium acetylhyaluronate, sodium hyaluronate, sodium polyaspartate (commercially available as AQUADEW SPA-30 from Ajinomoto), sodium polyglutamate (commercially available as AJICOAT SPG from Ajinomoto), sorbeth 20, sorbeth 6, sorbitol, trehalose, triglycerol, trimethyolpropane, tris (hydroxymethyl) amino methane salts, xylitol, xylose, and mixtures thereof.

Non-limiting examples of sunscreens which are useful in the compositions include 4-N,N-(2-ethylhexyl)methylaminobenzoic acid ester of 2,4-dihydroxybenzophenone, 4-N,N-(2-ethylhexyl)methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone, 4-N,N-(2-ethylhexyl)-methylaminobenzoic acid ester of 4-(2-hydroxyethoxy)dibenzoylmethane, 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomethyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, and mixtures thereof. Other useful sunscreens include aminobenzoic acid (PABA), benzylidene camphor, butyl methoxy dibenzoyl methane, diethanolamine p-methoxycinnamate, dioxybenzone, ethyl dihydroxypropyl (PABA), glyceryl aminobenzoate, homomethyl salicylate, isopropyl dibenzoyl methane, lawsone and dihydroxyacetone, menthyl anthranilate, methyl anthranilate, methyl benzylidene camphor, octocrylene, octyl dimethyl (PABA), octyl methoxycinnamate, oxybenzone, 2-phenylbenzimidazole-5-sulfonic acid, red petrolatum, sulisobenzone, titanium dioxide, triethanolamine salicylate, zinc oxide, and mixtures thereof. Exact amounts of sunscreens which can be employed will vary depending upon the sunscreen chosen and the desired Sun Protection Factor (SPF) to be achieved.

Anti-Acne Actives:

Non-limiting examples of useful anti-acne actives include the keratolytics such as salicylic acid (o-hydroxybenzoic acid), derivatives of salicylic acid such as 5-octanoyl salicylic acid and 4 methoxysalicylic acid, and resorcinol; retinoids such as retinoic acid and its derivatives (e.g., cis and trans); sulfur-containing D and L amino acids and their derivatives and salts, particularly their N-acetyl derivatives, a preferred example of which is N-acetyl-L-cysteine; lipoic acid; antibiotics and antimicrobials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxyisopropanol, ethyl acetate, clindamycin and meclocycline; sebostats such as flavonoids and bioflavonoids; bile salts such as scymnol sulfate and its derivatives, deoxycholate, and cholate; abietic acid; adapalene; allantoin; aloe extracts; arbietic acid and its salts; aryl-2,4 dioxo oxazolidine derivatives; ASEBIOL (available from Laboratories Serobiologiques, located in Somerville, N.J.); azaleic acid; barberry extracts; bearberry extracts; belamcanda chinensis; benzoquinolinones; benzoyl peroxide; berberine; BIODERMINE (available from Sederma, located in Brooklyn, N.Y.); bioflavinoids; bisabolol; S-carboxymethyl cysteine; carrot extracts; cassin oil; clove extracts; citral; citronellal; climazole; Completech MBAC-OS (available from Lipo); CREMOGEN M82 (available from Dragoco, located in Totowa, N.J.); cucumber extracts; dehydroacetic acid and its salts; dehydroeplandersterone salicylate; dichlorophenyl imidazoldioxolan which is commercially available as COMPLETECH MBAC-OS (from Lipo, located in Paterson, N.J.); DL valine and its esters; DMDM hydantoin; Epicutin TT (available from CLR); erythromycin; escinol; ethyl hexyl monoglyceryl ether; ethyl 2-hydroxy undecanoate; farnesol; farnesol acetate; geranoil; glabridin; gluconic acid; gluconolactone; glyceryl monocaprate; glycolic acid; grapefruit seed extract; gugu lipid; Hederagenin (available from Maruzen); hesperitin; hinokitol; hops extract; hydrogenated rosin; 10 hydroxy decanoic acid; ichtyhol; interleukin 1 alpha antagonists; iodo-2-propynyl butyl carbamate; Kapilarine (available from Greentech); ketoconazole; lactic acid; lemon grass oil; Lichochalcone LR15 (available from Maruzen); linoleic acid; LIPACIDE C8CO (available from Seppic, located in Paris, France); lovastatin; 4 methoxysalicylic acid; metronidazole; minocycline; mukurossi; neem seed oil; vitamin B₃ compounds (such as niacinamide and nicotinic acid); nisin; 5-octanoly salicylic acid; octopirox; panthenol; 1-pentadecanol; peonia extract; peppermint extract; phelladendron extract; 2-phenyl-benzothiophene derivatives; phloretin; PHLOROGINE (available from Secma); phosphatidyl choline; proteolytic enzymes; quercetin; red sandalwood extract; resorcinol; rosemary extract; rutin; sage extract; salicin; salicylic acid; skull cap extract; siber hegner extract; siberian saxifrage extract; silicol; sodium lauryl sulfate; sodium sulfoacetamide; Sophora Extract (available from Maruzen); sorbic acid; sulfur; sunder vati extract; tea tree oil; tetracyline; tetra hydroabietic acid; thyme extract; tioxolone; tocopherol; trehalose 6-undecylenoate; 3 tridecene-2-ol; triclosan; tropolone; UNITRIENOL T27 (available from Unichem, located in Gouda, Netherlands); vitamin D3 and its analogs; white thyme oil; willow bark extract; wogonin; Ylang Ylang; zinc glycerolate; zinc linoleate; zinc oxide; zinc pyrithione; zinc sulfate and mixtures thereof.

Antimicrobial and Antifungal Actives:

Non-limiting examples of antimicrobial and antifungal actives include beta-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione; clotrimazole; alantolactone; isoalantolactone; alkanet extract (alaninin); anise; arnica extract (helenalin acetate and 11, 13 dihydrohelenalin); Aspidium extract (phloro, lucinol containing extract); barberry extract (berberine chloride); bay sweet extract; bayberry bark extract (myricitrin); benzalkonium chloride; benzethonium chloride; benzoic acid and its salts; benzoin; benzyl alcohol; blessed thistle; bletilla tuber; bloodroot; bois de rose oil; burdock; butyl paraben; cade oil; CAE (available from Ajinomoto, located in Teaneck, N.J.); cajeput oil; Cangzhu; capsicum frutescens extract; caraway oil; cascarilla bark (sold under the tradename ESSENTIAL OIL); cedarleaf oil; chamomille; chaparral; chlorhexidine gluconate; chlorophenesin; chlorxylenol; cinnamon oil; citronella oil; clove oil; Crinipan AD (available from Climbazole); 2,3-dihydro-farnesol; dehydroacetic acid and its salts; dill seed oil; DOWICIL 200 (available from Dow Chemical, located in Midland, Mich.); echinacea; elenolic acid; epimedium; ethyl paraben; Fo—Ti; galbanum; garden bumet; GERMALL 115 and GERMALL II (available from ISP-Sutton Labs, located in Wayne, N.J.); German chamomile oil; giant knotweed; GLYDANT (available from Lonza, located in Fairlawn, N.J.); GLYDANT PLUS (available from Lonza); grapefruit seed oil; 1,6 hexanediol; hexamidine diisethionate; hinokitiol; honey; honeysuckle flower; hops; immortelle; iodopropynl butyl carbamide (available from Lonza); isobutyl paraben; isopropyl paraben; JM ACTICARE (available from Microbial Systems International, located in Nottingham, NG); juniper berries; KATHON CG (available from Rohm and Haas, located in Philadelphia, Pa.); kojic acid; labdanum; lavender; lemon balm oil; lemon grass; methyl paraben; mint; mume; mustard; myrrh; neem seed oil; ortho phenyl phenol; olive leaf extract (available from Bio Botanica); parsley; patchouly oil; peony root; 1,2 pentandiol; PHENONIP (available from Nipa Labs, located in Wilmington, Del.); phenoxyethanol; phytosphingosine; pine needle oil; PLANSERVATIVE (available from Campo Research); propyl paraben; purslane; quillaira; rhubarb; rose geranium oil; rosemary; sage; salicylic acid; sassafras; savory; sichuan lovage; sodium meta bisulfite; sodium sulfite; SOPHOLIANCE (available from Soliance, located in Compiegne, France); sorbic acid and its salts; sphingosine; stevia; storax; sucrose esters; tarmic acid; tea; tea tree oil (cajeput oil); thyme; triclosan; triclocarban; tropolone; turpentine; umbelliferone (antifungal); yucca; and mixtures thereof.

Anti-Wrinkle, Anti-Skin Atrophy and Skin Repair Actives:

Other anti-wrinkle actives may also be combined with exemplary cosmetic compositions disclosed herein. Non-limiting examples of anti-wrinkle and anti-skin atrophy actives include retinoic acid and its derivatives (e.g., cis and trans); retinal; retinol; retinyl esters such as retinyl acetate, retinyl palmitate, and retinyl propionate; vitamin B₃ compounds (such as niacinamide and nicotinic acid), salicylic acid and derivatives thereof (such as 5-octanoyl salicylic acid, heptyloxy 4 salicylic acid, and 4-methoxy salicylic acid); sulfur-containing D and L amino acids and their derivatives and salts, particularly the N-acetyl derivatives, a preferred example of which is N-acetyl-L-cysteine; thiols, e.g. ethane thiol; hydroxy acids, phytic acid, lipoic acid; lysophosphatidic acid; skin peel agents (e.g., phenol and the like); Actein 27-Deoxyactein Cimicifugoside (available from Cirnigoside); adapalene; ademethionine; adenosine; aletris extract; alkyl glutathione esters; alkoxyalkoxy alkoxyn benzoic and derivatives; aloe derived lectins; amino propane phosphoric acid; 3-aminopropyl dihydrogen phosphate; Amadorine (available from Barnet Products); anise extracts; AOSINE (available from Secma); arginine amino benzoate; ASC III (available from E. Merck, located in Darmstadt, Germany); ascorbic acid; ascorbyl palmitate; asiatic acid; asiaticosides; ARLAMOL GEO™ (available from ICI, located in Wilmington, Del.); azaleic acid; benzoic acid derivatives; bertholletia extracts; betulinic acid; BIOCHANIN A AND BIOPEPTIDE CL (available from Sederma, located in Brooklyn, N.Y.); BIOPEPTIDE EL (available from Sederma); biotin; blackberry bark extract; blackberry lily extracts; black cohosh extract; blue cohesh extract; butanoyl betulinic acid; carboxymethyl 1,3 beta glucan; catecholamines; chalcones; citric acid esters; chaste tree extract; clover extracts; coumestrol; CPC Peptide (available from Barnet Products); daidzein; dang gui extract; darutoside; debromo laurinterol; 1-decanoyl-glycero-phosphonic acid; dehydrocholesterol; dehydrodicreosol; dehydrodieugenol; dehydroepiandersterone; DERMOLECTINE (available from Sederma); dehydroascorbic acid; dehydroepiandersterone sulfate; dianethole; dihydroxy benzoic acid; 2,4 dihydroxybenzoic acid; diglycol guanidine succinate; diosgenin; disodium ascorbyl phosphate; dodecanedioic acid; Ederline (available from Seporga); Enderline (available from Laboratories Seporga); equol; eriodictyol; estrogen and its derivatives; ETF (available from Laboratories Seporga); ethocyn; ELESERYL SH (available from Laboratories Serobiologiques, located in Somerville, N.J.); ENDONUCLEINE (available from Laboratories Serobiologiques); ergosterol; eythrobic acid; fennel extract; fenugreek seed extract; FIBRASTIL (available from Sederma); FIBROSTIMULINES S and P (available from Sederma); FIRMOGEN LS 8445 (available from Laboratories Serobiologiques); formononetin; forsythia fruit extract; gallic acid esters; gamma amino butyric acid; GATULINE RC (available from Gattlefosse, located in Priest, France); genistein; genisteine; genistic acid; gentisyl alcohol; gingko bilboa extracts; ginseng extracts; ginsenoside (RO, R6-1, R6-2, R6-3, R_{C}, R_{D}, R_{E}, R_{F}, R_{F-2}, R_{G-1}, R_{G-2}); gluco pyranosyl-L-ascorbate; glutathione and its esters; glycitein; hesperitin; hexahydro curcumin; HMG-coenzyme A reductase inhibitors; hops extracts; 11 hydroxy undecanoic acid; 10 hydroxy decanoic acid; 25-hydroxycholesterol; 7-hydroxylated sterols; hydroxyethyl isostearyloxy isopropanolamine; hydroxy-tetra methyl piperidinyloxy; hypotaurine; ibukijakou extract; isoflavone SG 10 (available from Barnet Products); kinetin; kohki extract; L-2-OXO-thiazolidine-4-carboxylic acid esters; lactate dehydrogenase inhibitors; 1-lauryl, -lyso-phosphatidyl choline; lectins; lichochalcone LF15 (available from Maruzen); licorice extracts; lignan; lumisterol; lupenes; luteolin; lysophosphitidic acid; margin; melatonin; melibiose; metalloproteinase inhibitors; methoprene; methoprenic acid; mevalonic acid; MPC COMPLEX (available from CLR); N methyl serine; N methyl taurine; N,N.sup.1-bis(lactyl) cysteamine; naringenin; neotigogenin; o-desmethylangoiensin; oat beta glucan; oleanolic acid; pantethine; phenylalanine; photoanethone; piperidine; placental extracts; pratensein; pregnenolone; pregnenolone acetate; pregnenolone succinate; premarin; quillaic acid; raloxifene; REPAIR FACTOR 1 and REPAIR FACTOR FCP (both available from Sederna); retinoates (esters of C2-C20 alcohols); retinyl glucuronate; retinyl linoleate; S-carboxymethyl cysteine; SEANAMINE FP (available from Laboratories Serobiologiques); sodium ascorbyl phosphate; soya extracts; spleen extracts; tachysterol; taurine; tazarotene; tempol; thymulen; thymus extracts; thyroid hormones; tigogenin; tocopheryl retinoate; toxifolin; traumatic acid; tricholine citrate; trifoside; uracil derivatives; ursolic acid; vitamin D3 and its analogs; vitamin K; vitex extract; yam extract; yamogenin; zeatin; and mixtures thereof.

Skin Barrier Repair Actives:

Skin barrier repair actives are those skin care actives that can help repair and replenish the natural moisture barrier function of the epidermis. Non-limiting examples of skin barrier repair actives include Alpha Lipid (available from Lucas Meyer); ascorbic acid; biotin; biotin esters; brassicasterol; caffeine; campesterol; canola derived sterols; Cennamides (available from Ennagram); Ceramax (available from Alban Muller); CERAMAX (available from Quest, located in Ashford, England); CERAMIDE 2 and CERAMIDE HO3™ (both available from Sederma); CERAMIDE II (available from Quest); CERAMIDE III and IIIB (both available from Cosmoferm, located in Deft, Netherlands); CERAMIDE LS 3773 (available from Laboratories Serobiologiques); CERAMINOL (available from Inocosm); Cerasol and Cephalip (both available from Pentapharm); cholesterol; cholesterol hydroxystearate; cholesterol isostearate; 7 dehydrocholesterol; DERMATEIN BRC and DERMATEIN GSL (both available from Hormel); ELDEW CL 301 AND ELDEW PS 203 (both available from Ajinomoto); Fitobroside (available from Pentapharm); galactocerebrosides; Generol 122 (available from Henkel); glyceryl serine amide; hydroxyethyl isostearyl isopropanolamine; lactic acid; Lactomide (available from Pentapharm); lanolin; lanolin alcohols; lanosterol; lauric acid N laurylglucamide; lipoic acid; N-acetyl cysteine; N-acetyl-L-serine; N-methyl-L-Serine; Net Sterol-ISO (available from Barnet Products); vitamin B3 compounds (such as niacinamide and nicotinic acid); palmitic acid; panthenol; panthetine; phosphodiesterase inhibitors; PHYTO/CER (available from Intergen); phytoglycolipid millet extract (available from Barnet Products Distributer, located in Englewood, N.J.); PHYTOSPHINGOSINE (available from Gist Brocades, located in King of Prussia, Pa.); PSENDOFILAGGRIN (available from Brooks Industries, located in South Plainfield, N.J.); QUESTAMIDE H (available from Quest); serine; sigmasterol; sitosterol; soybean derived sterols; sphingosine; sphingomylinase; S-lactoyl glutathione; stearic acid; Structurine (available from Silah); SUPER STEROL ESTERS (available from Croda); thioctic acid; THSC CERAMIDE OIL (available from Campo Research); trimethyl glycine; tocopheryl nicotinate; vitamin D3; Y2 (available from Ocean Pharmaceutical); and mixtures thereof.

Cosmetic Soothing Actives:

Cosmetic soothing actives can be effective in preventing or treating inflammation of the skin. Non-limiting examples of cosmetic soothing agents include the following categories: propionic acid derivatives; acetic acid derivatives; fenamic acid derivatives; biphenylcarboxylic acid derivatives; and oxicams. Non-limiting examples of useful cosmetic soothing actives include acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, bucloxic acid, absinthium, acacia, aescin, alder buckthorn extract, allantoin, aloe, APT (available from Centerchem), arnica, astragalus, astragalus root extract, azulene, Baicalin SR 15 (available from Barnet Products Dist.), baikal skullcap, baizhu, balsam canada, bee pollen, BIOPHYTEX (available from Laboratories Serobiologiques), bisabolol, black cohosh, black cohosh extract blue cohosh, blue cohosh extract, boneset, borage, borage oil, bradykinin antagonists, bromelain, calendula, calendula extract, Canadian Willowbark Extract (available from Fytokem), candelilla wax, Cangzhu, canola phytosterols, capsicum, carboxypeptidase, celery seed, celery stem extract, CENTAURIUM (available from Sederma), centaury extract, chamazulene, chamomile, chamomile extract, chaparral, chaste tree, chaste tree extract, chickweed, chicory root, chicory root extract, chirata, chishao, collodial oatmeal, comfrey, comfrey extract, CROMOIST CM GLUCAN (available from Croda), darutoside, dehurian angelica, devil's claw, divalent metals (such as, magnesium, strontium, and manganese), doggrass, dogwood, Eashave (available from Pentapharm), eleuthero, ELHIBIN (available from Pentapharm), ENTELINE 2 (available from Secma), ephedra, epimedium, esculoside; ethacrynic acid, evening primrose, eyebright, Extract LE-100 (available from Sino Lion), Fangfeng, feverfew, ficin, forsythia fruit, Fytosterol 85 (available from Fytokem), ganoderma, gaoben, Gatuline A (available from Gattefosse), gentian, germanium extract, gingko bilboa extract, ginkgo, ginseng extract, goldenseal, gorgonian extract, gotu kola, grape fruit extract, guaiac wood oil, guggal extract, helenalin esters, henna, honeysuckle flower, horehound extract, horsechestnut, horsetail, huzhang, hypericum, ichthyol, immortelle, ipecac, job's tears, jujube, kola extract, LANACHRYS 28 (available from Lana Tech), lemon oil, lianqiao, licorice root, ligusticum, ligustrum, lovage root, luffa, mace, magnolia flower, manjistha extract, margaspidin, matricin, melatonin, MICROAT IRC (available from Nurture), mints, mistletoe, Modulene (available from Seporga), mono or diglucosides of glabridin, mono or diglucosides of gentisin, MTA (5′-deoxy-5′-methylhioadenosine), mung bean extract, musk, N-methyl arginine, oat beta glucan, oat extract, orange, panthenol, papain, phenoxyacetic acid, peony bark, peony root, Phytoplenolin (available from Bio Botanica), phytosphingosine, Preregen (available from Pentapharm), purslane, QUENCH T (available from Centerchem), quillaia, red sage, rehmannia, rhubarb, rosemary, rosmarinic acid, royal jelly, rue, rutin, sandlewood, sanqi, sarsaparilla, saw palmetto, SENSILINE (available from Silab), SIEGESBECKIA (available from Sederma), stearyl glycyrrhetinate, Stimutex (available from Pentapharm), storax, strontium nitrate, sweet birch oil, sweet woodruff, tagetes, tea extract, thyme extract, tienchi ginseng, tocopherol, tocopheryl acetate, triclosan, turmeric, urimei, ursolic acid, white pine bark, witch hazel xinyi, yarrow, yeast extract, yucca, and mixtures thereof.

Artificial Tanning Actives and Accelerators:

Non-limiting examples of artificial tanning agents and accelerators include dihydroxyacetaone; tyrosinel; tyrosine esters such as ethyl tyrosinate and glucose tyrosinate; acetyl tyrosine; phospho-DOPA; brazilin; caffeine; coffee extracts; dihydroxyacetone; DNA fragments; isobutyl methyl xanthine; methyl xanthine; Phototan (available from Laboratoires Serobiologiques); prostaglandins; tea extracts; theophylline; tyrosine; UNIPERTAN P2002 and UNIPERTAN P27 (both available from Unichem); and mixtures thereof.

Skin Lightening Actives:

Skin lightening actives can decrease the amount of melanin in the skin or provide an such an effect by other mechanisms. Non-limiting examples of skin lightening actives useful herein include adapalene, aloe extract, alpha-glycaryl-L-ascorbic acid, aminotyroxine, ammonium lactate, anethole derivatives, apple extract, arbutin, areca catechu L. extract, ascorbic acid, ascorbyl palmitate, azelaic acid, bamboo extract, bearberry extract, bletilla tuber, bupleurum falcatum extract, burnet extract, Burnet Power (available from Barnet Products), butyl hydroxy anisole, butyl hydroxy toluene, butyl resoreinol, Chuanxiong, cola decaballo extract, Dang-Gui, deoxyarbutin, 1,3 diphenyl propane derivatives, 2,5 dihydroxybenzoic acid and its derivatives, 2-(4-acetoxyphenyl)-1,3 dithane, 2-(4-hydroxyphenyl)-1,3 dithane, ellagic acid, escinol, estragole derivatives, esculoside, esculetin, FADEOUT (available from Pentapharm), Fangfeng, fennel extract, gallic acid and its derivatives, ganodenna extract, gaoben, GATULINE WHITENING (available from Gattlefosse), genistic acid and its derivatives, gentisyl alcohol, glabridin and its derivatives, gluco pyranosyl-1-ascorbate, gluconic acid, glucosamine, glycolic acid, glycyrrhizinic acid, green tea extract, 4-Hydroxy-5-methyl-3[2H]-furanone, hydroquinine, 4 hydroxyanisole and its derivatives, 4-hydroxy benzoic acid derivatives, hydroxycaprylic acid, hyptis extract, inositol ascorbate, kojic acid, kojic dipalnitate, lactic acid, lemon extract, licorice extract, Licorice P-TH (available from Barnet Products), linoleic acid, Melfade (available from Pentapharm), MELAWHITE (available from Pentapharm), Melanostatine DM (available from Laboratories Seporga), morus alba extract, mulberry root extract, niacinamide, 5-octanoyl salicylic acid, parsley extract, phellinus linteus extract, pinon blanco extract, pinon negro extract, piri-piri extract, pyrogallol derivatives, retinoic acid, retinol, retinyl esters (acetate, propionate, palmitate, linoleate), 2,4 resorcinol derivatives, 3,5 resorcinol derivatives, rose fruit extract, rucinol, salicylic acid, Song-Yi extract, Sophora Powder (available from Barnet Products), 4-thioresorein, 3,4,5 trihydroxybenzyl derivatives, tranexamic acid, tyrostat (Rumex Extract available from Fytokem), Tyroslat 10,11 (available from Fytokem), vanilla derivatives, vitamin D3 and its analogs, and mixtures thereof.

Sebum Stimulators:

Sebum stimulators can increase the production of sebum by the sebaceous glands. These skin care actives are especially useful for post menopausal women who are sebum deficient. Non-limiting examples of sebum stimulating actives include bryonolic acid, completech MBAC-DS, dehydroetiandrosterone (also known as DHEA), orizanol and mixtures thereof.

Sebum Inhibitors:

Sebum inhibitors can decrease the production of sebum by the sebaceous glands. Non-limiting examples of sebum inhibiting actives include aluminium hydroxy chloride, ASEBIOL (available from Laboratories Serobiologiques), BIODERMINE (available from Sederma), climbazole, COMPLETECH MBAC-OS (available from Lipo), corticosteroids, cucumber extracts, dehydroacetic acid and its salts, dichlorophenyl imidazoldioxolan (available from Elubiol), gugulipiu, ketoconazole, Lichochalcone LR 15 (available from Maruzen), niacinamide, phloretin, PHLOROGINE (available from Secma), Phycosaccharide Anti-Acne (available from Codif), S-carboxylmethyl cysteine, sepicontrol AS, spironolactone, tioxolone, tocopherol, tranexamic acid, UNITRIENOL T27 (available from Unichem), zincidone (UC1B), and mixtures thereof.

Protease Inhibitors:

Non-limiting examples of protease inhibitors which are useful in the exemplary cosmetic compositions are selected from the group consisting of A E Complex (available from Barnet Products); ALE (available from Seporga); allicin; alpha lupaline; Aosaine (available from Secma); Aprotinin (available from Pentapharm); areca catechu (Betel Nut) extract; areca catechu extracts; Blue Algae Extract (available from Collaborative Labs); Centaurium (available from Sederma); cholesterol sulfate; CMST (available from Bioetica); Dermoprotectine (available from Sederma); Disacoside HF 60 (available from Barnet Products); Elhibin (available from Pentapharm); Fluid Out Colloid (available from Vegetech); Hypotaurine (available from Sogo Pharmaceutical); In Cyte Heathes (available from Collaborative Labs); Micromerol (available from Collaborative Labs); Pefabloc SP (available from Pentapharm); Sepicontrol AS (available from Seppic); Siegesbeckia (available from Sederma); Sophorine (available from Barnet Products); Thiotaine (available from Barnet Products); uncaria gambis roxburgh extract; zinc and mixtures thereof.

Skin Tightening Agents:

The cosmetic compositions may also include skin-tightening agents as active ingredients. Non-limiting examples of skin tightening agents which are useful in the compositions of the present invention are those selected from the group consisting of Biocare SA (available from Amerchol); egg albumen; Flexan 130 (available from National Starch); Gatuline Lifting (available from Gattefosse); Pentacare HP (available from Pentapharm); Vegeseryl (available from Laboratories Serobioloques) and mixtures thereof.

Viscosity Modifiers:

Suitable thickeners and viscosity modifiers include water-soluble polyacrylic and hydrophobically modified polyacrylic resins such as Carbopol and Pemulen, starches such as corn starch, potato starch, tapioca, gums such as guar gum, gum arabic, cellulose ethers such as hydroxypropyl cellulose, hydroxyethyl cellulose, carboxymethyl cellulose, and the like.

Emulsifiers:

A wide variety of emulsifiers are useful herein and include, but not limited to, sorbitan esters, glyceryl esters, polyglyceryl esters, methyl glucose esters, sucrose esters, ethoxylated fatty alcohols, hydrogenated castor oil ethoxylates, sorbitan ester ethoxylates, polymeric emulsifiers, silicone emulsifiers, glyceryl monoesters, preferably glyceryl monoesters of C16-C22 saturated, unsaturated and branched chain fatty acids such as glyceryl oleate, glyceryl monostearate, glyceryl monopalmitate, glyceryl monobehenate, and mixtures thereof; polyglyceryl esters of C16-C22 saturated, unsaturated and branched chain fatty acids, such as polyglyceryl-4 isostearate, polyglyceryl-3 oleate, diglycerol monooleate, tetraglycerol monooleate and mixtures thereof; methyl glucose esters, preferably methyl glucose esters of C16-C22 saturated, unsaturated and branched chain fatty acids such as methyl glucose dioleate, methyl glucose sesquiisostearate, and mixtures thereof; sucrose fatty acid esters, preferably sucrose esters of C12-C22 saturated, unsaturated and branched chain fatty acids such as sucrose stearate, sucrose trilaurate, sucrose distearate (e.g., CRODESTA® F10, and mixtures thereof; C12-C22 ethoxylated fatty alcohols such as oleth-2, oleth-3, steareth-2, and mixtures thereof; hydrogenated castor oil ethoxylates such as PEG-7 hydrogenated castor oil; sorbitan ester ethoxylates such as PEG-40 sorbitan peroleate, Polysorbate-80, and mixtures thereof; polymeric emulsifiers such as ethoxylated dodecyl glycol copolymer; and silicone emulsifiers such as laurylmethicone copolyol, cetyldimethicone, dimethicone copolyol, and mixtures thereof.

Humectants:

Non-limiting examples of humectants include glycerin, propylene glycol, and laminaria digitata extract.

Further agents, which may be used, will be described in the following examples of various compositions and their manufacturing procedure in accordance with the teachings described herein.

The exemplary embodiments of the skin care compositions disclosed herein may also be used alone or in combination with one or more products. For instance, a skin care system may include, but is not limited to, a day cream, night cream, eye serum, mask, cleanser, and an exfoliant. The following examples are directed to various exemplary embodiments of the skin care composition and methods of manufacturing those compositions.

### EXAMPLE 1

The composition of this example includes sun-screening agents and is preferably used as a daytime facial cream, and includes sunscreen. The ingredients included in the composition are listed in Table 1:

TABLE 1

| Seq. | INCI Name | Trade Name | % w/w |
|---|---|---|---|
| 1 | Water | Water | 55.49 |
| 2 | Carbomer | Carbopol 940 | 0.40 |
| 3 | Glycerin | Glycerin 99% | 1.00 |
| 3 | Trisodium EDTA | Sequestrene NA3T | 0.10 |
| 3 | Methylparaben | Methylparaben | 0.25 |
| 3 | Propylparaben | Propylparaben | 0.10 |
| 4 | PEG-8/SMDI Copolymer | Polyoprepolymer - | 1.00 |
| | | 15 | |
| 5 | Octyldodecyl Neopentanoate | Elefac I 205 | 5.00 |
| 5 | Octinoxate | Parsol MCX | 7.50 |
| 5 | Oxybenzone | Benzophenone-3 | 3.00 |
| 5 | Glyceryl Stearate (1.2500%) PEG-100 Stearate | Lipomulse 165 | 2.50 |
| | (1.2500%) | | |
| 5 | Cetyl Alcohol | Lipocol C | 1.50 |
| 5 | Caprylic/Capric Triglyceride | Captex 300 | 2.00 |
| 5 | Octisalate | Escalol 587 | 5.00 |
| 5 | Cetearyl Alcohol (1.4000%) Ceteareth-20 | Lipowax D | 1.50 |
| | (0.6000%) | | |
| 5 | Tricaprin | Captex 1000 | 0.50 |
| 5 | Dimethicone | Dow Corning | 0.25 |
| | | 200/100 cts Fluid | |
| 5 | Dipalmitoyl Hydroxyproline | Sepilift DPHP | 1.00 |
| | | (Seppic) | |
| 6 | Water | Water | 1.00 |
| 6 | Triethanolamine | TEA 99% | 0.40 |
| 7 | Phenoxyethanol | Phenoxyethanol | 1.00 |
| 8 | Caprylic/Capric Triglyceride (and) Cucumis Melo | Natural Melon | 1.50 |
| | (Melon) Extract (and) Musa Sapientum (Banana) | Blend XX-000981 | |
| | Fruit Extract (and) Vitis Vinifera (Grape) Fruit | (Ungerer) | |
| | Extract (and) Iris Florentina Root Extract (and) | | |
| | Hibiscus Abelmoschuus | | |
| 9 | Water (and) Hyaluronic Acid | Hyasol BT | 1.00 |
| | | (Centerchem) | |
| 9 | Acetyl Hexapeptide 3 | Argireline | 5.00 |
| | | (Centerchem) | |
| 9 | Water (1.7440%) Butylene Glycol (0.1550%) | APT (Centerchem) | 2.00 |
| | Ahnfeltia Concinna Extract (0.0410%) | | |
| | Glycosaminoglycans (0.0600%) | | |
| 9 | Water (0.0097%) Lecithin (0.0001%) Magnesium | Oxysomes (Barnet) | 0.01 |
| | Ascorbyl Phosphate (0.0001%) Tocopherol (0.0001%) | | |

With reference to Table 1, the composition of Example 1 is manufactured using a stainless steel, jacketed main kettle, equipped with sweep agitation and a Lightning® mixer. The Seq. #1 water is heated to approximately 78-80° C. under mixing or agitation. Under good agitation, the Seq. #2 Carbomer powder is slowly added by sprinkling slowly and evenly across the agitating water. Care should be taken not to aerate the batch. The batch is then checked to assure that it is free from any lumps, then cooled to approximately 76-78° C. The remaining Seq. #3 materials are added to the batch under adequate agitation. Just prior to the oil phase addition of the Seq. #5 materials, the Seq. #4 Copolymer is added to the water phase batch in the main tank under good agitation. In a stainless steel side kettle, the Seq. #5 materials are combined and heated to approximately 80-82° C., under high agitation. At proper temperatures, the Seq. #5 combination is added to the Seqs. 1, 2, 3, and 4 materials in main tank with high agitation. The temperature and agitation is maintained until the emulsion is stabilized. Thereafter, the batch is cooled to approximately 70° C. and the agitation setting is switched to sweep as the batch thickens to avoid air entrapment. At approximately 70° C., the premixed Seq. #6 materials are added, and the batch is cooled to approximately 42° C. At approximately 42° C., the Seq. #7 and then the Seq. #8 materials are added, mixing uniformly between additions. The Seq. #9 materials are then added, one at a time, being sure batch is uniform before proceeding. Thereafter, the batch is cooled to approximately 25° C. A standard may be established by producing several batches which are similar (e.g. three batches). Once a standard is made, top and bottom samples may be taken from each subsequent batch for comparison to the standard.

### EXAMPLE 2

The composition of this example is preferably used as a nighttime facial cream and includes the following ingredients as listed in Table 2:

TABLE 2

| Seq | INCI Name | Trade Name | % w/w |
|---|---|---|---|
| 1 | Water (Aqua) | Water | 53.84 |
| 2 | Carbomer | Carbopol 940 | 0.35 |
| 3 | Glycerin | Glycerin 96% | 3.00 |
| 3 | Trisodium EDTA | Sequestrene NA3T | 0.10 |
| 3 | PEG-8/SMDI Copolymer | Polyoprepolymer-15 | 1.00 |
| 3 | Methylparaben | Methylparaben | 0.25 |
| 3 | Propylparaben | Propylparaben | 0.10 |
| 4 | Isostearyl Palmitate | Protachem ISP | 3.00 |
| 4 | Glyceryl Stearate (1.2500%) PEG-100 Stearate | Lipomulse 165 | 2.50 |
| | (1.2500%) | | |
| 4 | Squalane | Squalane | 2.00 |
| 4 | Cetyl Alcohol | Lipocol C | 1.00 |
| 4 | Caprylic/Capric Triglyceride | Captex 300 | 10.00 |
| 4 | Cetearyl Alcohol (1.4000%) Ceteareth-20 | Lipowax D | 2.00 |
| | (0.6000%) | | |
| 4 | Cetyl Lactate | Ceraphyl 28 | 0.50 |
| 4 | Dimethicone | Dow Corning 200/100 cts | 0.50 |
| | | Fluid | |
| 4 | Dipalmitoyl Hydroxyproline | Sepilift DPHP (Seppic) | 1.00 |
| 5 | Water | Water | 1.00 |
| 5 | Triethanolamine | TEA 99% | 0.35 |
| 6 | Polyacrylamide (0.1700%) C13-14 Isoparaffin | Sepigel 305 | 0.25 |
| | (0.0625%) Laureth-7 (0.0175%) | | |
| 7 | Cyclomethicone | Dow Corning 345 Fluid | 1.00 |
| 8 | Caprylic/Capric Triglyceride (and) Cucumis | Natural Melon Blend | 1.50 |
| | Melo (Melon) Extract (and) Musa Sapientum | XX-000981 | |
| | (Banana) Fruit Extract (and) Vitis Vinifera | (Ungerer) | |
| | (Grape) Fruit Extract (and) Iris Florentina | | |
| | Root Extract (and) Hibiscus Abelmoschuus | | |
| 8 | Cucumis Melo (Melon) Extract | Crodarome Liquid Melon | 0.50 |
| | | Extract (Croda) | |
| 9 | Phenoxyethanol | Phenoxyethanol | 1.00 |
| 9 | Water (and) Hyaluronic Acid | Hyasol 1% (Centerchem). | 1.00 |
| 9 | Acetyl Hexapeptide 3 | Argireline (Centerchem) | 10.00 |
| 9 | Imperata Cylindrica (Root) Extract (0.1000%) | Moist 24 (Croda) | 0.25 |
| | Water (0.0800%) Glycerin (0.0550%) PEG-8 | | |
| | (0.0125%) Carbomer (0.0025%) | | |
| 9 | Water (1.7440%) Butylene Glycol (0.1550%) | APT (Centerchem) | 2.00 |
| | Ahnfeltia Concinna Extract (0.0410%) | | |
| | Glycosaminoglycans (0.0600%) | | |
| 9 | Water (0.0097%) Lecithin (0.0001%) Magnesium | Oxysomes (Barnet) | 0.01 |
| | Ascorbyl Phosphate (0.0001%) Tocopherol | | |
| | (0.0001%) | | |

With reference to Table 2, the composition of Example 2 is prepared by heating the Seq. #1 water in a stainless steel, jacketed main kettle, equipped with sweep agitation and a Lightnin® mixer, to approximately 80-82° C. while mixing. Under high agitation, the Seq. #2 carbomer powder is sprinkled slowly and evenly across the water. Care should be taken not to aerate the batch. Alternatively, the carbomer powder may be added prior to heating the water. The Seq. #3 materials are then added to the main kettle, and the batch is mixed well. Once the batch is free from lumps, it is cooled to 76-78° C. The Seq. #4 materials are combined in a stainless steel side kettle, under high agitation, and heated to approximately 80-82° C. The Seq. #4 mixture is then slowly added to the batch in the main kettle under adequate agitation, maintaining the energy and temperature until the emulsion is well formed and stabalized. Once the emulsion is well formed and stabilized, the batch is allowed to cool to approximately 70° C. At 70° C. the premixed Seq. #5 materials are then added to the batch. The mixing type and speed are adjusted as necessary as the batch thickens. The Seq. #6 material is then added in the same manner. The batch is then cooled to 50° C. and the Seq. #7 material is added to the batch. The batch is cooled to 42° C. The Seq. #8 materials are then added, one at a time, mixing well between additions, and reaching uniformity before proceeding. The batch is then cooled to approximately 35° C. The Seq. #9 materials are then added one at a time, mixing well between additions, and reaching uniformity. The batch is then cooled to room temperature, and is preferably compared to a standard.

### EXAMPLE 3

The composition of this example is preferably used as an eye serum and includes the following ingredients as listed in Table 3:

TABLE 3

| Seq. | INCI Name | Trade Name | % w/w |
|---|---|---|---|
| 1 | Water (Aqua) | Deionized Water | 52.71 |
| 2 | Carbomer | Carbopol 940 (BF Goodrich) | 0.30 |
| 3 | Glycerin (14.3800%) Water (5.6000%) | Zilgel VV (Presperse) | 20.00 |
| | Sodium Polyacrylate (0.0200%) | | |
| 4 | Water | Deionized Water | 2.00 |
| 4 | Triethanolamine | TEA 99% | 0.28 |
| 5 | Butylene Glycol | Butylene Glycol | 2.00 |
| 5 | Ethoxydiglycol | Transcutol (Gattefosse) | 0.50 |
| 5 | Salix Alba (Willow) Bark Extract | NAB Willowbark Extract | 0.20 |
| | | #09020 (Arch) | |
| 5 | Hexapeptide-3 | Argireline (Centerchem) | 10.00 |
| 5 | Water (0.9094%) Propylene Glycol | Remoduline (Silab/Rita) | 1.25 |
| | (0.3125%) Citrus Aurantium Amara (Bitter | | |
| | Orange) Flower Extract (0.0281%) | | |
| 5 | Sodium Hyaluronate | Hyasol BT (Centerchem) | 1.00 |
| 5 | Water (0.0097%) Lecithin (0.0001%) | Oxysomes (Barnet) | 0.01 |
| | Magnesium Ascorbyl Phosphate (0.0001%) | | |
| | Tocopherol (0.0001%) | | |
| 6 | Polysorbate 20 | Tween 20 (ISI) | 1.50 |
| 6 | Phenoxyethanol (0.7200%) Methylparaben | Phenonip (Nipa) | 1.00 |
| | (0.1600%) Propylparaben (0.0350%) | | |
| | Ethylparaben (0.0350%) Butylparaben | | |
| | (0.0500%) | | |
| 6 | Polyethylene | CL-2080 (Kobo) | 1.00 |
| 6 | Isononyl Isononanoate | Dermol 99 (Alzo) | 0.25 |
| 6 | Dimethicone Copolyol | Dow Corning 193 (Dow | 1.00 |
| | | Corning) | |
| 6 | Isostearyl Neopentanoate | Ceraphyl 375 (ISP) | 0.50 |
| 6 | Neopentyl Glycol Dicaprylate/Dicaprate | Liponate NPGC-2 (Lipo) | 0.50 |
| 6 | Cyclomethicone | Dow Corning 345 (Dow | 1.00 |
| | | Corning) | |
| 7 | Water (1.7440%) Butylene Glycol (0.1550%) | APT GC (Centerchem) | 2.00 |
| | Ahnfeltia Concinna Extract (0.0410%) | | |
| | Glycosaminoglycans (0.0600%) | | |
| 7 | Water (0.3200%) Imperata Cylindrica (Root) | Moist-24 (Croda) | 1.00 |
| | Extract (0.4000%) Gylcerin (0.2200%) PEG- | | |
| | 8 (0.0500%) Carbomer (0.0100%) | | |

With reference to Table 3, the composition of Example 3 is prepared by heating the Seq. #1 water in a stainless steel jacketed kettle equipped with a Lightning® mixer and sweep agitation, to approximately 80-82° C. while agitating to eliminate any possible presence of Gram Positive bacteria. The water is then allowed to slowly cool, and at around 40° C. the Seq. #2 Carbomer is slowly added. The batch is then agitated well and mixed it is lump-free and uniform. Top and bottom samples may be quality checked before proceeding. The Seq. #3 material is then added, switching to sweep as batch thickens to prevent aeration. The batch is allowed to continue cooling. In a side phase stainless steel kettle, the Seq. #4 materials are premixed and added to the batch. The Seq. #5 materials are then added, one at a time, mixing well and preferably reaching uniformity in between additions. In side phase stainless steel kettle, the Seq. #6 materials are premixed and added to the batch. The Seq. #7 materials are added, one at a time, and the batch is mixed until uniform. The batch is allowed to cool to room temperature, and top and bottom samples are taken for comparison to a standard.

### EXAMPLE 4

The composition of this example is preferably used as a facial mask and includes the following ingredients as listed in Table 4:

TABLE 4

| Seq. | INCI Name | Trade Name | % w/w |
|---|---|---|---|
| 1 | Water (Aqua) | Water | 76.80 |
| 1 | Methylparaben | Methylparaben | 0.25 |
| 1 | Disodium EDTA | Dissolvine Na2 (Akzo) | 0.10 |
| 1 | Glycerin | Glycerin 99% | 3.00 |
| 2 | Chondrus Crispus | Viscarin SD 389 (FMC) | 0.30 |
| 2 | Chondrus Crispus | Viscarin GP 209 (FMC) | 0.10 |
| 3 | Hydroxyethylcellulose | Natrosol 250HHR | 1.00 |
| | | (Hercules/Aqualon) | |
| 4 | Hypnea Musciformis Extract (0.4800%) | Ormagel SH (ISI) | 1.00 |
| | Gellidiela Acerosa Extract (0.1000%) | | |
| | Sargassum Filipendula Extract (0.2400%) | | |
| | Sorbitol (0.1600%) water (0.0200%) | | |
| 4 | Butylene Glycol (0.2300%) Water (0.2450%) | Complex EPX-246 BG Anti- | 0.50 |
| | Humulus Lupulus (Hops) Extract (0.0050%) | Aging (ISI) | |
| | Rosmarinus Officinalis (0.0050%) Equisetum | | |
| | Arvense Extract (0.0050%) Pinus Sylvestris | | |
| | Cone Extract (0.0050%) Citrus Medica | | |
| | Limonum (Lemon) Fruit Extract (0.0050%) | | |
| 4 | Hydrolyzed Soy Protein | Phytokine (Coletica/ | 0.1 |
| | | Englehart) | |
| 4 | Water, Ahnfeltia Concinna Extract, | APT-GC (Centerchem) | 2.00 |
| | Glycosaminoglycans | | |
| 4 | Hydrolyzed Wheat Protein | Sitolene (Silab/Rita) | 0.10 |
| 4 | Zea Mays (Corn) Extract | Deliner (Coletica Inc.) | 2.00 |
| 4 | Acetyl Hexapeptide-3 | Argireline (Centerchem) | 5.00 |
| 4 | Propylene Glycol (1.4530%) Water | Complex 4-Oxygen | 2.00 |
| | (0.4000%) Butylene Glycol (0.0750%) Vitis | (Centerchem) | |
| | Vinifera (Grape) Leaf Extract (0.0520%) | | |
| | Glycine Soya (Soybean) Germ Extract | | |
| | (0.0100%) Triticum Vulgare (Wheat) Germ | | |
| | Extract (0.0100%) | | |
| 5 | Phenoxythanol | Phenoxythanol | 1.00 |
| 5 | Cucumis Melo (Melon) Extract | Crodarome Liquid Melon | 1.00 |
| | | (Croda) | |
| 5 | PPG-5 Ceteth-20 | Procetyl A WS (Croda) | 1.00 |
| 6 | Water (Aqua) | Water | 1.00 |
| 6 | Benzoic Acid | Benzoic Acid | 0.25 |
| 6 | Hydrolyzed Wheat Protein/PVP Crosspolymer | Hydrotriticum PVP (Croda) | 2.50 |

With reference to Table 4, the composition of Example 4 may be prepared by heating the Seq. #1 materials in a jacketed, stainless steel kettle equipped with a variable speed Lightnin® type mixer, to approximately 80° C. while mixing well, to dissolve the methylparaben. The Seq. #2 materials are then added one at a time, into the kettle, under adequate Lightnin® type agitation. Once the batch becomes uniform, the materials of Seq. #3 are added as in the previous step. The batch is then cooled to approximately 40° C., switching to sweep setting as the batch thickens, and being careful not to aerate the batch. At approximately 40° C., the Seq. #4 materials are added, one at a time, mixing well in between additions. The batch should preferably be homogeneous prior to each addition. The Seq. # 5 material is then added in the same manner. In a side kettle equipped with Lightnin® type agitation, the Seq. #6 materials are premixed and warmed to 40° C., then added to the batch in the same manner. The batch is mixed until uniform. Top and bottom standards are preferably taken for comparison to a standard.

### EXAMPLE 5

The composition of this example may be used as a cleanser and includes the following ingredients as listed in Table 5:

TABLE 5

| Seq. | INCI Name | Trade Name | % W/W |
|---|---|---|---|
| 1 | Water (Aqua) | Water | 62.369 |
| 1 | Glycerin | Glycerin 99% | 4.750 |
| 1 | Disodium EDTA | Dissolvine Na 2 (Akzo) | 0.100 |
| 1 | Methylparaben | Methylparaben | 0.250 |
| 1 | Propylparaben | Propylparaben | 0.100 |
| 2 | Chondrus Crispus (Carrageenan) | Viscarin SD 389 (FMC | 0.200 |
| | | Corp) | |
| 3 | Acrylates/C10-30 Alkyl Acrylate Crosspolymer | Carbopol 2020 (Protameen/ | 0.250 |
| | | BF Goodrich) | |
| 4 | Olive Oil PEG-7 Esters | Olivem 300 (Presperse) | 0.500 |
| 4 | Tridecyl Stearate (3.5200%) Neopentyl Glycol | Lipovol MOS-70 (Lipo) | 8.000 |
| | Dicaprylate/Dicaprate (3.5200%) Tridecyl | | |
| | Trimellitate (0.9600%) | | |
| 4 | Cetearyl Alcohol (1.2950%) Ceteareth-20 | Lipowax D (Lipo) | 1.850 |
| | (0.5550%) | | |
| 4 | PEG-4 Olivate | Olivem 700 (Presperse) | 1.850 |
| 4 | Glyceryl Stearate SE | Lipo 470 SE (Lipo) | 3.750 |
| 4 | Dimethicone | Dow 200 Fluid 350 c/s | 0.010 |
| | | (Dow Corning) | |
| 5 | Polyacrylamide (0.6800%) C13-14 Isoparaffin | Sepigel 305 (Seppic) | 1.000 |
| | (0.2500%) Laureth-7 (0.0700%) | | |
| 6 | Cocamidopropyl Betaine | Cocamidopropyl Betaine | 6.000 |
| | | (Lexaine C) | |
| 7 | Phenoxyethanol | Phenoxyethanol | 1.000 |
| 8 | Glyceryl Polymethacrylate | Lubragel NP (ISP) | 2.000 |
| 9 | Caprylic/Capric Triglyceride (and) Cucumis | Natural Melon Blend | 2.000 |
| | Melo (Melon) Extract (and) Musa Sapientum | XX-000981 | |
| | (Banana) Fruit Extract (and) Vitis Vinifera | (Ungerer) | |
| | (Grape) Fruit Extract (and) Iris Florentina Root | | |
| | Extract (and) Hibiscus Abelmoschuus | | |
| 9 | Cucumis Melo (Melon) Extract | Liquid Melon Crodarome | 1.000 |
| | | (Croda) | |
| 9 | Water (0.0088%) Butylene Glycol (0.0007%) | APT GC (Centerchem) | 0.010 |
| | Ahnfeltia Concinna Extract (0.0002%) | | |
| | Glycosaminoglycans (0.0003%) | | |
| 9 | Isododecane | Permethyl 99A (Presperse) | 3.000 |
| 9 | Vitis Vinifera (Grape) Seed Extract (0.0009%) | Leucocyandin Phytosome | 0.001 |
| | Phospholipids (0.0001%) | (Indena - Lipo) | |
| 9 | Acetyl Hexapeptide-3 | Argireline (Centerchem) | 0.010 |

With reference to Table 5, the composition of Example 5 is prepared by heating the combined Seq. #1 materials in a stainless steel, jacketed main kettle, equipped with sweep agitation and a Lightnin® mixer to approximately 78-80° C. under Lightnin® mixing. Under high agitation, the Seq. #2 material is then sprinkled slowly and evenly across the agitating batch. Care should be taken not to aerate the batch. The batch is then checked to make sure that it is lump free and cooled to approximately 76-78° C. The Seq. #3 material is then added in a similar manner. In a stainless steel side kettle with high agitation, the Seq. #4 materials are combined and heated to approximately 80-82° C. At proper temperatures, the Seq. #4 batch is slowly added to the main kettle, while maintaining high agitation and temperature, until the emulsion is well formed and stabilized. The batch is then cooled to approximately 70° C., switching to sweep as the batch thickens. At approximately 70° C., the Seq. #5 material is added. This material should be well mixed prior to adding, since it may separate in storage. At this point it may be necessary to increase the mixing speed until the batch is smooth and uniform. The prior mixing speed may then be resumed, or the sweep setting may be adjusted accordingly. The Seq. #6 material then added and the batch is cooled to approximately 42° C. At approximately 42° C. the Seq. #7 material is added. The Seq. #8 material is then added, being sure that the material is evenly dispersed before continuing. The batch is then cooled to 35° C. and the Seq. #9 materials are added, one at a time, or a premix is created for addition. The batch is then mixed until uniform and cooled to 25° C. Top and bottom samples may be taken and compared to a standard.

### EXAMPLE 6

The composition of this example may be used as an exfoliant, and includes the following ingredients as listed in Table 6.

TABLE 6

| Seq. | INCI Name | Trade Name | % W/W |
|---|---|---|---|
| 1 | Water (Aqua) | Water | 57.275 |
| 1 | Glycerin | Glycerin 99% | 4.750 |
| 1 | Disodium EDTA | Dissolvine NA2 (Akzo) | 0.100 |
| 1 | Methylparaben | Methylparaben | 0.250 |
| 1 | Propylparaben | Propylparaben | 0.100 |
| 2 | Chondrus Crispus (Carrageenan) | Viscarin SD 389 (FMC | 0.250 |
| | | Corp) | |
| 3 | Xanthan Gum | Keltrol CG (Kelco) | 0.150 |
| 4 | Olive Oil PEG-7 Esters | Olivem 300 (Presperse) | 0.500 |
| 4 | Tridecyl Stearate (3.5200%) Neopentyl Glycol | Lipovol MOS-70 (Lipo) | 10.000 |
| | Dicaprylate/Dicaprate (3.5200%) Tridecyl | | |
| | Trimellitate (0.9600%) | | |
| 4 | Cetearyl Alcohol (2.1000) Ceteareth-20 | Lipowax D (Lipo) | 3.000 |
| | (0.9000) | | |
| 4 | PEG-4 Olivate | Olivem 700 (Presperse) | 1.850 |
| 4 | Glyceryl Stearate SE | Lipo 470 SE (Lipo) | 3.750 |
| 4 | Dimethicone | Dow 200 Fluid 350 c/s | 0.050 |
| | | (Dow Corning) | |
| 5 | Polyacrylamide (0.5100%) C13-14 Isoparaffin | Sepigel 305 (Seppic) | 0.750 |
| | (0.1875%) Laureth-7 (0.0525%) | | |
| 6 | Disodium Laureth Sulfosuccinate (0.9000%) | Chemoryl SFB-2 | 6.000 |
| | Cocamidopropyl Hydroxysultaine (0.6000%) | (Chemron) | |
| | Myristamine Oxide (0.3300%) Glycerin | | |
| | (0.3300%) Sodium Cocoyl Glutamate | | |
| | (0.0600%) PEG-150 Distearate (0.0600%) | | |
| | water (3.7200%) | | |
| 7 | Phenoxyethanol | Phenoxyethanol | 1.000 |
| 7 | Methylchloroisothiazolinone, | Kathon CG (Rohm and | 0.100 |
| | Methylisothiazolinone | Haas) | |
| 8 | Glyceryl Polymethacrylate | Lubragel NP (ISP) | 2.000 |
| 9 | Caprylic/Capric Triglyceride (and) Cucumis | Natural Melon Blend | 1.000 |
| | Melo (Melon) Extract (and) Musa Sapientum | XX-000981 | |
| | (Banana) Fruit Extract (and) Vitis Vinifera | (Ungerer) | |
| | (Grape) Fruit Extract (and) Iris Florentina Root | | |
| | Extract (and) Hibiscus Abelmoschuus | | |
| 9 | Cucumis Melo (Melon) Extract | Liquid Melon Crodarome | 1.000 |
| | | (Croda) | |
| 10 | Water (0.0088%) Butylene Glycol (0.0007%) | APT GC (Centerchem) | 0.010 |
| | Ahnfeltia Concinna Extract (0.0002%) | | |
| | Glycosaminoglycans (0.0003%) | | |
| 10 | Isododecane | Permethyl 99A (Presperse) | 3.000 |
| 10 | Vitis Vinifera (Grape) Seed Extract (0.0009%) | Leucocyanidins Phytosome | 0.001 |
| | Phospholipids (0.0001%) | (Indena - Lipo) | |
| 11 | Acetyl Hexapeptide-3 | Argireline (Centerchem) | 0.010 |
| 11 | Salix Alba (Willow) Bark Extract | NAB Willow bark Extract | 1.000 |
| | | #09020 (Arch) | |
| 11 | Hydrogenated Jojoba Wax | Ice White Jojoba Spheres | 2.000 |
| | | 40/60 (Desert Whale) | |
| 11 | Synthetic Iron Oxide, Oxidized Polyethylene | Accuscrub S-200600 (Grey) | 0.100 |
| | Homopolymer | (Accutech) | |

With reference to Table 6, the composition of Example 6 is prepared by heating the combined Seq. #1 materials in a stainless steel, jacketed main kettle, equipped with sweep agitation and a Lightnin® mixer to approximately 78-80° C. under Lightnin® mixing. The Seq. #2 material is then sprinkled slowly and evenly across the agitating batch, under propeller agitation. Care should be taken not to aerate the batch. The batch is then checked to make sure that it is lump free and cooled to approximately 76-78° C. The Seq. #3 material is then added in a similar manner. In a stainless steel side kettle with good agitation, the Seq. #4 materials are combined and heated to approximately 80-82° C. At proper temperatures, the Seq. #4 batch is slowly added to the main kettle, while maintaining high agitation and temperature, until the emulsion is well formed and stabilized. The batch is then cooled to approximately 70° C., switching to sweep as the batch thickens. At approximately 70° C., the Seq. #5 material is added. This material should be well mixed prior to adding, since it may separate in storage. At this point it may be necessary to increase the mixing speed until batch is smooth and uniform. The prior mixing speed may then be resumed, or the speed may be adjusted accordingly. The batch is then cooled to approximately 42° C., and the Seq. #6 then the Seq. #7 materials are added. The Seq. #8 material is then added, being sure that the material is evenly dispersed before continuing. The batch is then cooled to approximately 42° C. and the aromatic Seq. #9 materials are added, one at a time, mixing until the batch is uniform in between additions. The batch is then cooled to approximately 35° C. The Seq. #10 materials are then added, one at a time, mixing well with each addition. The batch is mixed until uniform. The Seq. #11 materials are then added, and the batch is mixed until the particles are evenly distributed. The batch is allowed to cool to 25° C. and top and bottom samples may be taken and compared to a standard.

### EXAMPLE 7

The composition of this example is preferably used as a skin refining primer and includes the following ingredients as listed in Table 7:

TABLE 7

| Seq. | INCI Name | Trade Name | % w/w |
|---|---|---|---|
| 1 | Water (Aqua) | Deionized Water | 83.195 |
| 1 | Glycerin | Glycerin | 3.000 |
| 1 | Disodium EDTA | Dissolvine Na2 (Akzo) | 0.10 |
| 1 | Aloe Barbadensis Leaf Juice | Aloe Barbadensis Leaf Juice | 1.00 |
| | | (Lily of the Desert) | |
| 1 | Sodium PCA | Ajidew N-50 (Ajinomoto) | 0.500 |
| 1 | Methylparaben | Methylparaben | 0.250 |
| 1 | Benzophenone-4 | Benzophenone-4 | 0.050 |
| 1 | Butylene Glycol | Butylene Glycol | 3.000 |
| 2 | Hydroxyethylcellulose | Natrosol HHR | 0.750 |
| | | (Hercules/Aqualon) | |
| 3 | Salix Alba (Willow)Bark Extract | NAB Willowbark Extract | 3.500 |
| | | #09020 (Arch) | |
| 4 | Lavendula Angustifolia (Lavender) oil | Lavender Oil (Ungerer) | 0.020 |
| 4 | Propylparaben | Propylparaben | 0.020 |
| 4 | PPG-5-Ceteth-20 | Procetyl AWS (Croda) | 1.500 |
| 5 | Water (and) butylene glycol (and) Camellia | Actiphyte of Green Tea BG50 | 0.100 |
| | Oleifera Leaf Extract | (Active Organics) | |
| 6 | Phenoxyethanol | Phenoxyethanol | 1.000 |
| 7 | Water (1.7440%) Butylene Glycol | APT GC (Centerchem) | 2.000 |
| | (0.1550%) Ahnfeltia Concinna Extract | | |
| | (0.0410%) Glycosaminoglycans | | |
| | (0.0600%) | | |
| 8 | Water (and) Red No. 40 (CI 16035) | Red No. 40(1% Aq.Sol) | 0.015 |

With reference to Table 7, the composition of Example 7 is prepared by heating the Seq. #1 materials in a stainless steel jacketed kettle equipped with a Lightning® mixer and sweep agitation, to approximately 80° C. while mixing to solublize the methylparaben and eliminate any gram Positive bacteria that may be present. The water is then cooled to 45° C. During the cooling process, the Seq. #2 material may be slowly incorporated to the kettle, while under adequate agitation. Top and bottom samples from the batch are checked for lumps before proceeding. The Seq. #3 material is then added. In a stainless steel side kettle equipped with Lightnin® type agitation, the Seq. #4 materials are premixed. It may be necessary to warm the mixture to approximately 40-42° C. in order to obtain clarity. Very slowly, and under high energy, the Seq. #4 mixture is added to the main batch. The Seq. #4 mixture is preferably added in small intervals, wherein the batch is allowed to clear before additions, in order to obtain a clear batch. The Seq. #5, 6, and 7 materials are added in the same manner, one at a time, being sure that the batch is clear and uniform in between additions. The Seq. #8 material is then added to the batch. Top and bottom samples may be taken for comparison to a standard.

### EXAMPLE 9

The composition of this example may be used as a moisturizer or hydrating serum and includes the following ingredients as listed in Table 9:

TABLE 9

| Seq. | INCI Name | Trade Name | % W/W |
|---|---|---|---|
| 1 | Water | Water | 66.72 |
| 1 | Disodium EDTA | Dissolvine Na2 (Akzo) | 0.10 |
| 1 | Methylparaben | Methylparaben | 0.10 |
| 2 | Carrageenan (Chondrus Crispus) | Viscarin SD 389 (FMC) | 1.40 |
| 3 | Glycerin, Glyceryl Polyacrylate | Hispagel 200 (Centerchem) | 14.00 |
| 4 | Water (1.7440%) Butylene Glycol | APT/GC (Centerchem) | 2.00 |
| | (0.1550%) Ahnfeltia Concinna | | |
| | Extract (0.0410%) | | |
| | Glycosaminoglycans (0.0600%) | | |
| 5 | Acetyl Hexapeptide-3 | Argireline (Centerchem) | 5.00 |
| 6 | Ceramide 3, Ceramide 6, Ceramide | Sk-Influx (Centerchem) | 5.00 |
| | 1, Cholesterol, Phytosphingosine, | | |
| | Sodium Lauroyl Lactylate, | | |
| | Carbomer, Xanthan Gum, | | |
| | Methylparaben, Propylparaben | | |
| 6 | Benzophenone-4 | Uvinul MS-40 (BASF) | 0.02 |
| 7 | Olive Oil Peg-7 Esters | Olivem 300 (Presperse) | 3.00 |
| 7 | Hyaluronic Acid | Hyasol 1% (Centerchem) | 1.00 |
| 7 | Phenoxyethanol | Phenoxyethanol | 1.00 |
| 7 | Caprylic/Capric Triglyceride, | Natural Melon Concentrate | 0.50 |
| | Cucumis Melo (Melon) Extract | #51226(Belle Aire) | |
| | Musa Sapientum (Banana) Fruit | | |
| | Extract, Vitis Vinifera (Grape) Fruit Extract, | | |
| | Iris Florentina | | |
| 8 | Water, Blue No. 1 (CI 42090) | Blue #1; 1.00% Aq. Sol | * QS |
| | | | (0.16) |

With reference to Table 9, the composition of Example 9 is prepared by combining the Sq. #1 materials in a stainless steel main kettle, equipped with a Lightnin® mixer, and heating to 80° C. while mixing. The batch is mixed just long enough to dissolve the paraben, and is then allowed to start cooling. The Seq. #2 carrageenan is added while the batch is still warm, being sure it is evenly dispersed and there are no clumps present. The batch is then cooled to approximately 35° C. Seqs. #3,4 and 5 are then added, one at a time, being sure that each is evenly dispersed before proceeding. Seqs. # 6 and 7 are then premixed and added to the main kettle. The batch is mixed until uniformity is obtained. A pre-determined amount of Seq. #8 may then be added for color to match a standard. Top and bottom samples may then be taken and compared to a standard.

### EXAMPLE 10

The composition of this example is preferably used as a hand cream and/or a night cream and includes the following ingredients as listed in Table 10:

TABLE 10

| Seq. | INCI Name | Trade Name | % W/W |
|---|---|---|---|
| 1 | Water (Aqua) | Water | 51.75 |
| 1 | Glycerin | Glycerin 99% | 2.00 |
| 1 | Methylparaben | Methylparaben | 0.25 |
| 1 | Disodium EDTA | Dissolvine Na@ (Akzo) | 0.10 |
| 1 | PEG-150 Distearate | Lipopeg 6000 DS (Lipo) | 0.25 |
| 2 | Xanthan Gum | Keltrol CG (Kelco) | 0.30 |
| 3 | Bulyrospermum (Shea) Fruit Butter | Shea Butter (Sederma) | 3.00 |
| 3 | Theobroma Cacao (Cocoa) Seed Butter | Cocoa Butter (Ruger) | 2.00 |
| 3 | Lignoceryl Erucate | Crodamol LGE (Croda) | 6.00 |
| 3 | Trideccyl Trimellitate | Liponate TDTM (Lipo) | 6.00 |
| 3 | Glyceryl Stearate, PEG-100 Stearate | Lipomulse 165 (Lipo) | 2.75 |
| 3 | Dipalmitoyl Hydroxproline | Sepilift DPHP (Seppic) | 1.00 |
| 3 | Cetearyl Alcohol | Crodacol CS-50 (Croda) | 0.50 |
| 3 | Cetyl Alcohol | Lipocol C (Lipo) | 1.00 |
| 3 | Emulsifying Wax | Polowax (Croda) | 3.00 |
| 3 | Propylparaben | Propylparaben | 0.10 |
| 3 | Dimethicone | Dow corning 200/350 (Dow | 1.00 |
| | | Corning) | |
| 4 | Mica (and) Silica | SM-2000 (Preserse) | 2.00 |
| 5 | Phenoxyethanol | Emmeressence 1160 Rose | 1.00 |
| | | (Cognis) | |
| 5 | Water (and) Salix Nigra (Willow) Bark | Nab Willow Bark Extract | 10.00 |
| | Extract | (Arch) | |
| 5 | Hexapeptide-10 | Scrilesine (Centerchem) | 2.00 |
| 5 | Acetyl Hexapeptide-3 | Argireline (Centerchem) | 2.00 |
| 5 | Caprylic/Capric Triglyceride (and) citrus | Aromatic grapefruit extract | 1.50 |
| | grandis (grapefruit) Peel extract | G4329 (Carrubba | |
| | | Fragrances) | |
| 5 | Ahnfeltia Concinna Extract, | APT-GC (Centerchem) | 0.50 |
| | Glycosaminoglycans, Butylene Glycol | | |

With reference to Table 10, the composition of Example 10 is prepared by combining the Seq. # 1 materials in a stainless kettle, equipped with sweep agitation and a propeller mixer, and heating to 80-82 degrees centigrade under propeller mixing. Under adequate propeller agitation, the Seq. #2 gum powder is slowly and evenly sprinkled across the agitating water, being careful not to aerate the batch. In stainless steel side kettle with good agitation, the seq. #3 materials are combined and heated to 80-82 degrees centigrade. The Seq.#3 batch is then added to Seq.'s # 1 and 2 under adequate agitation, maintaining agitation and temperature until an emulsion is well formed and stabilized. The main batch is then cooled to 50 degrees centigrade. The Seq. #4 powder is then added to the main batch by slowly sprinkling the powder on top of batch with a sifting-type motion. The mixing type and speed may be adjusted when necessary as the batch thickness. The batch is then cooled to 42 degrees centigrade, and the Seq. #5 materials are added, one at a time, mixing well with each addition. The batch is finally cooled to 25 degrees centigrade, and top and bottom samples are preferably compared to a standard.

### EXAMPLE 11

The composition of this example is preferably used as a cream for the neck and jawline and includes the following ingredients as listed in Table 11:

TABLE 11

| Seq. | INCI Name | Trade Name | % W/W |
|---|---|---|---|
| 1 | Water (Aqua) | Water | 43.55 |
| 2 | Carbomer | Glycerin 99% | 0.40 |
| 3 | Glycerin | Methylparaben | 3.00 |
| 3 | Disodium EDTA | TEA 99% | 0.10 |
| 3 | PEG-8/SMDI Copolymer | CMC 99-7MF | 1.00 |
| | | (Aqualon/Hercules) | |
| 3 | PVP | Veegum 1W (RT | 0.50 |
| | | Vanderbilt) | |
| 3 | Water (and) Salix Nigra (Willow) Bark | Kelirol CG (CP Kelco) | 5.00 |
| | Extract | | |
| 3 | Methylparaben | Pemulen TR-1 (BF | 0.25 |
| | | Goodrich) | |
| 3 | Propylparaben | Propylparaben | 00.10 |
| 4 | Isostearyl Palmitate | Protochem ISP (Protameen) | 3.00 |
| 4 | Glyceryl | Lipomulse 165 (Lipo) | 2.50 |
| | Stearate (and) | | |
| | PEG-100 Stearate | | |
| 4 | Squalane | Squalane (Barnet) | 2.00 |
| 4 | Cetyl Alcohol | Lipocol C (Lipo) | 1.00 |
| 4 | Caprylic/Capric Triglyceride | Liponate GC (Lipo) | 10.00 |
| 4 | Cetearyl Alcohol (and) Ceteareth-20 | Lipowax D (Lipo) | 3.00 |
| 4 | Stearic Acid | Stearic Acid (Lipo) | 1.00 |
| 4 | Cetyl Lactate | Ceraphyl 28 (ISP) | 0.50 |
| 4 | Dimethicone | Dow Corning 200/100 cts | 1.00 |
| | | Fluid(Dow | |
| | | Corning) | |
| 5 | Water | Water | 1.00 |
| 5 | Triethanolamine | TEA 99% | 0.35 |
| 6 | Polycrylamide (and) C13-14 Isoparaffin | Sepigel 305 (Seppic) | 0.75 |
| | (and) Laureth-7 | | |
| 7 | Cyclomethicone | Dow Corning 345 Fluid | 1.00 |
| | | (Dow Corning) | |
| 7 | Mica (and) Silica (and) Titanium Dioxide | Soft Vision 58557 | 3.00 |
| | (CI 77891) | (Presperse) | |
| 8 | Caprylic/Capric Triglyceride (and) | Natural Melon Blend | 1.50 |
| | Cucumis Melo (Melon) Extract (and) | XX-00981 | |
| | Musa Sapientum (Banana) Fruit Extract | | |
| | (and) Vitis Vinifera (Grape) Fruit Extract | | |
| | (and) Iris Florentina Root | | |
| | Extract (and) Hibiscus Abelmoschuus | | |
| 8 | Phenoxyethanol | Emmeressence 1160 Rose | 1.00 |
| | | (Cognis) | |
| 9 | Water (and) Hyaluronic Acid | IIyasol 1% (Centerchem) | 1.00 |
| 9 | Acetyl Hexapeptide-3 | Argeriline (Centerchem) | 2.00 |
| 9 | Palmitoyl Tripeptide-3 (and) Glycerin | Syn-Coll (Centerchem) | 2.00 |
| 9 | Hydrolyzed Soy Protein | Phytokine (Coletica) | 2.00 |
| 9 | Hexapeptide-3 | Serilesine (Centerchem) | 2.00 |
| 9 | Ceramide 3 (and) Ceramide 6 (and) | Sk-Influx (Centerchem) | 3.00 |
| | Ceramide 1 (and) Phytosphingosine (and) | | |
| | Cholesterol (and) Sodium Lauroyl Lactylate | | |
| | (and) Carbomer (and) Xanthan Gum | | |
| 9 | Ahnfeltia Concinna Extract, | APT-GC (Centerchem) | 2.00 |
| | Glycosaminoglycans, Butylene Glycol | | |

With reference to Table 11, the composition of Example 11 is prepared by combining the Seq. # 1 materials in a jacketed main kettle, equipped with sweep agitation and a propeller mixer, and heating to 80-82 degrees centigrade under propeller mixing. Under adequate propeller agitation, the seq. #2 Carbomer powder is slowly added by sprinkling slowly and evenly across agitating water, being careful not to aerate the batch. For production purposes, the carbomer may be added prior to heating the water. The Seq. #3 materials are then added to the main kettle in the water phase while mixing well. The batch is then checked to make sure that it is free from lumps, and cooled to cooled to 76-78 degrees centigrade. The Seq. #4 materials are combined in a stainless steel side kettle under good agitation, and heated to 80-82 degrees centigrade. At proper temperatures, the Seq.#4 batch is slowly added to the main kettle under adequate agitation. The temperature and agitation is maintained until an emulsion is well formed and stabilized. The batch is then cooled to 70 degrees centigrade. At 70 C, the premixed Seq. #5 materials are added to the batch. The mixing type and speed are adjusted when necessary, as the batch thickens. The Seq. #6 material is added in the same manner, and the batch is cooled to 50 degrees C. Seq.#7 materials are then added, one at a time, wherein the second material, which is a powder, is added by slowly sprinkling on top of the batch with a sifting-type motion. The batch is then cooled to 42 C. At 42 C degrees centigrade, the Seq.#8 materials are slowly added, one at a time, mixing well between additions, and reaching uniformity before proceeding. The batch is then cooled to 35 C. At 35 C, the Seq.#9 materials are added, one at a time, mixing well between additions. The batch is then cooled to 25 degrees centigrade, and top and bottom samples are preferably compared to a standard.

### EXAMPLE 12

The composition of this example is preferably used as a foundation and includes the following ingredients as listed in Table 12:

TABLE 12

| Seq. | INCI Name | Trade Name | % W/W |
|---|---|---|---|
| 1 | Water (Aqua) | Water | 37.15 |
| 1 | Butylene Glycol | Glycerin 99% | 5.00 |
| 1 | Methylparaben | Methylparaben | 0.20 |
| 1 | Triethanolamine | TEA 99% | 1.50 |
| 2 | Sodium Carboxymethylcellulose | CMC 99-7MF | 0.30 |
| | | (Aqualon/Hercules) | |
| 2 | Magnesium Aluminum Silicate | Veegum 1W (RT | 0.35 |
| | | Vanderbilt) | |
| 2 | Xanthan Gum | Kelirol CG (CP Kelco) | 0.25 |
| 2 | Acrylates/CiO-30 Alkyl Acrylate | Pemulen TR-1 (BF | 0.15 |
| | Crosspolymer | Goodrich) | |
| 3 | Titaniun Dioxide (CI 77891) | Titanium Dioxide 3328 | 12.00 |
| | | (Universal Preserv-A-Chem) | |
| 3 | Iron Oxides (CI 77491, 77492) | Yellow hon Oxide 33-130 | 1.20 |
| | | (Sun Chemical) | |
| 3 | Iron Oxides (CI 77491) (and) | Ultra Hydrophobic Red | 0.13 |
| | Triethoxycaprylylsilane (and) | iron Oxide P2124 (LCW) | |
| | Diniethicone | | |
| 3 | Iron Oxide (CI 77499) (and) | Black Iron Oxide AS R0423 | 0.02 |
| | Triethoxycaprylylsilane | (LCW) | |
| 3 | Iron Oxides | fronOxideBrown33-115 (Sun | 0.40 |
| | | Chemical) | |
| 3 | Titanium Dioxide (CI 77891) (and) Iron Oxides | Photolite PK-S (Presperse) | 2.00 |
| | (CI 77491, 77492) | | |
| 3 | Mica (and) Silica | SM-2000 (Presperse) | 2.00 |
| 4 | Hydrogenated Lecithin | Lecinol S-10 (Barnet) | 0.30 |
| 4 | Isoeicosane | Permethyl 102A (Presperse) | 10.00 |
| 4 | Isostearic Acid | Liponate ISA (Lipo) | 1.00 |
| 4 | Stearic Acid | Stearic Acid (Lipo) | 0.50 |
| 4 | Glyceryl Stearate (and) PEG-100 Stearate | Lipomulse 165 (Lipo) | 2.25 |
| 4 | Tridecyl Trimellitate | Liopnate TDTM (Lipo) | 1.00 |
| 4 | Dimethicone | Dow Coming 200/100 (Dow | 1.00 |
| | | Corning) | |
| 4 | Propylparaben | Propylparaben | 0.20 |
| 5 | Cyclomethicone (and) Polysilicone-1 1 | Gransil GCM (Grant | 10.00 |
| | | Industries) | |
| 6 | Phenoxyethanol | Emmeressence 1160 Rose | 1.00 |
| | | 1.00(Cognis) | |
| 6 | Water (and) Butylene (Glycol (and) Ahnfeltia | APT GC (Centerchem) | 2.00 |
| | Concinna Extract (and) Glycosaminoglycans | | |
| 6 | Hexapeptide-10 | Serilesine (Centerchem) | 2.00 |
| 6 | Acetyl Hexapeptide-3 | Argeriline (Centerchem) | 5.00 |
| 6 | Caprylic/Capric Triglyceride (and) | Natural Melon Blend | 1.00 |
| | Cucumis Melo (Melon) Extract (and) Musa | Aroma XX-000 981 | |
| | Sapientum (Banana) Fruit Extract | (Ungerer) | |
| | (and) Vitis Vinifera (Grape) Fruit Extract | | |
| | (and) Iris Florentina Root Extract (and) | | |
| | Hibiscus Abelmoschuus | | |
| 7 | Mica (and) Titanium Dioxide (CI 77891) | Timiron MP 149 | 0.10 |
| | | (EMD/Rona) | |

With reference to Table 12, the composition of Example 12 is prepared by combining the Seq. # 1 materials in a jacketed stainless kettle, equipped with homogenization, sweep and a propeller type mixer, and heating to 78-80 degrees centigrade. The Seq. #2 gums are slowly added, one at a time, under homogenization and maintaining the temperature, until the batch is uniform and free of lumps. The Seq. #3 colorants are then added, one at a time, under homogenization, making sure they are fully dispersed before proceeding. Top and bottom samples are preferably checked. In stainless steel side kettle equipped with propeller agitation, the seq. #4 materials are added and heated to 80-82 degrees centigrade. The Seq. #4 batch is slowly added to the main batch under homogenization until an emulsion is well formed and stabilized, at which point the mixing type is switched to propeller, and the batch is cooled to 50 degrees centigrade. At 45-50 C, the Seq. #5 Gel is added to batch. It may be necessary to switch to sweep agitation after incorporation of Seq. #5. The batch is then cooled to 42 degrees centigrade, and the Seq. #6 and Seq. #7 materials are added, one at a time, mixing well between additions, and reaching uniformity before proceeding. The batch is then cooled to 25 degrees centigrade, and top and bottom samples are preferably compared to a standard.

### EXAMPLE 13

The composition of this example is preferably used as a nighttime hand lotion and includes the following ingredients as listed in Table 13:

TABLE 13

| Seq. | INCI Name | Trade Name | % W/W |
|---|---|---|---|
| 1 | Water (Aqua) | Water, Deionized | 42.46 |
| 1 | Glycerin | Glycerin 99% | 2.00 |
| 1 | Disodium EDTA | Dissolvine NA2 (Akzo) | 0.10 |
| 1 | Methylparaben | Methylparaben | 0.25 |
| 1 | Propylparaben | Propylparaben | 0.10 |
| 1 | PEG-8/SMDI Copolymer | Polyoprepolymcr-15 (Barnet) | 1.00 |
| 2 | Carbomer | Carbopol 940 | 0.20 |
| | | (Noveon/Protameen) | |
| 3 | Chondrus Crispus (Carrageenan) | Viscarin SD 389 (FMC/Earth | 0.05 |
| | | Supplied Products) | |
| 4 | Bisabolol | Bisabolol(Lipo). | 0.02 |
| 4 | Neopentyl Glycol | Liponate NPGC-2 (Lipo) | 1.00 |
| | Dicaprylate/Dicaprate | | |
| 4 | Octyldodecyl Neopentanoate | Elefac I-205 (Alzo/Bernel) | 2.50 |
| 4 | Octisalate | Escalol 587 (ISP) | 3.50 |
| 4 | Octinoxate | Escalol 557 (ISP) | 7.50 |
| 4 | Butyl Methoxydibenzoylmethane | Parsol 1789 (Roche) | 2.00 |
| 4 | Benzophenone-3 | Escalol 567 (ISP) | 3.50 |
| 4 | PEG-4 Olivate | Olivem 700 (Presperse) | 2.50 |
| 4 | Glyceryl Stearate (and) PEG-100 | Lipomulse 165 (Lipo) | 2.00 |
| | Stearate | | |
| 4 | Squalane | Squalane (Centerchem) | 3.00 |
| 4 | Cetyl Lactate | Ceraphyl28 (ISP) | 2.00 |
| 4 | Butyrospermum Parkii (Shea Butter) | Shea Butter (Sederma) | 0.50 |
| | Fruit | | |
| 4 | Cetyl Alcohol | Lipocol C (Lipo) | 0.60 |
| 4 | Cetearyl Alcohol (and) Ceteareth-20 | Lipowax D (Lipo) | 1.00 |
| 4 | Hydrogenated Lecithin | Lecinol S-10 (Barnet) | 0.30 |
| 5 | Water | Water | 1.00 |
| 5 | Triethanolamine | TEA 99% | 0.17 |
| 6 | Polyacrylamide (and) C13-14 | Sepigel 305 (Seppic) | 0.50 |
| | Isoparaffin (and) Laureth-7 | | |
| 7 | Titanium Dioxide (and) Silica (and) | Presperse Soft Vision | 2.50 |
| | Mica | (Presperse) | |
| 7 | Mica (and) Silica | SM 2000 (Presperse) | 2.00 |
| 8 | Caprylic/Capric Triglyceride (and) citrus grandis | Aromatic grapefruit extract | 1.75 |
| | (grapefruit) Peel extract | G4329 (Carrubba Fragrances) | |
| 9 | Salix Alba (Willow) Bark Extract | Nab Willow Bark Extract | 5.00 |
| | | (Arch) | |
| 9 | Palmitoyl Tripeptide 3 (and) Glycerin | Syn-Coll (Centerchem) | 2.00 |
| 9 | Hexapeptide-10 | Serilesine (Centerchem) | 2.00 |
| 9 | Acetyl Hexapeptide-3 | Argireline (Centerchem) | 2.00 |
| 9 | Water, Ahnfeltia Concinna Extract, | APT (Centerchem) | 2.00 |
| | Glycosaminoglycans | | |
| 9 | Phenoxyethanol | Emmer essence 1160 Rose | 1.00 |
| | | (Cognis) | |

With reference to Table 13, the composition of Example 13 is prepared combining the Seq. # 1 materials in stainless steel jacketed kettle equip with propeller mixer and sweep agitation, under propeller mixing and heating to 78-80 degrees centigrade. The Seq. # 4 materials are combined in a stainless steel jacketed side kettle equip with a propeller mixer and heated to 80-82 Centigrade, while mixing. The batch must be mixed while heating, and care must be taken not to overheat, as the Lecinol S-10 will burn. While maintaining mixing, the Seq. #2 carbomer is added to the main water phase at 78 degrees centigrade (or ambient temperature for manufacturing purposes if easier) under good agitation and heated when ready until the parabens are dissolved and the Carbomer forms a uniform, lump free solution, lump free. Top and bottom samples may be quality checked at this point before proceeding. The Seq. #3 carrageenan is added in the sample processing format as was the carbomer. A quality check may also be done at this point. The temperature is then decreased to 74-76 degrees centigrade. At proper temperatures, the Seq. #4 materials are slowly added to the main kettle, maintaining temperature and agitation until the emulsion is stabilized. The batch is then cooled to 70 degrees centigrade. At 70 degrees centigrade the premixed Seq. #5 materials are added to the batch. Thereafter, also at 70 degrees centigrade the Seq. #6 Sepigel is added to the batch with good agitation and mixing until the batch appearance is smooth. (The drum should be rolled just prior to pre-weighing and also prior to addition, as this material separates upon standing). It may be necessary to adjust the mixing speed or switch to side sweep during this addition, whenever necessary. The Seq. #7 powder is slowly sifted/sprinkled in, being sure not to clump mixture. The batch is then cooled to 50 degrees C. At 50 degrees C., the Seq. #7 powders are sprinkled in very slowly across the top of the batch as to not have materials clumping. The batch is then cooled to 40-42 C, switching to sweep agitation as necessary. At 40-42 degrees Centigrade the Seq. # 8 Fragrance is added and incorporated uniformly. The batch is allowed to continue cooling to 30-35 Degrees Centigrade. At 30-35 Degrees Centigrade, the Seq. # 9 materials are added one at a time, being sure that the batch is mixed to uniformity each time before continuing with further additions. Top and bottom samples are then compared to a standard.

### EXAMPLE 14

The composition of this example is preferably used as a sunscreen cream for the face and body. The ingredients included in the composition are listed in Table 14:

TABLE 14

| Seq. | INCI Name | Trade Name | % W/W |
|---|---|---|---|
| 1 | Water (Aqua) | Water | 56.98 |
| 1 | Pentylene Glycol | Hydrolite-5 (Dragoco) | 5.00 |
| 2 | Xanthan Gum | Keltrol CG (Kelco) | 0.50 |
| 3 | Zinc oxide | Z-Cote (BASF) | 2.00 |
| 4 | Sodium Dihydroxycetyl Phosphate | Dragophos S (Dragoco) | 3.00 |
| 4 | Glyceryl Stearate SE | Dracorin GMS SE (Dragoco) | 2.00 |
| 4 | Beeswax (Cera Alba) | White Beeswax (Hansen) | 1.00 |
| | Cetearyl Alcohol (and) Ceteareth-20 | Lipowax D (Lipo) | 1.50 |
| 4 | Ethylhexyl Ethylhexanoate | Dra oxat EH (Dragoco) | 8.00 |
| 4 | Polyglyceryl-3 Dicaprate | Tegosoft PC31 (Goldschmidt) | 0.30 |
| 4 | Triisononanoin | Isodragol (Dragoco) | 4.00 |
| 4 | Bisabolol | Dragosantol (Dragoco) | 0.10 |
| 4 | Meridamate | Dermoblock MA (Alzo) | 3.50 |
| 4 | Octinoxate | Escalol 557 (ISP) | 7.50 |
| 4 | Oxybenzone | Eusolex 4360 (RonalMerck) | 2.00 |
| 4 | Polyacrylamide (and) C13-14 | Sepigel 305 (Seppic) | 1.00 |
| | Isoparaffin (and) Laureth 7 | | |
| 5 | Capryhc/Capnc Triglyceride (and) | Super Natural Melon Blend | 0.50 |
| | Cucumis Melo (Melon) Extract | Concentrate Fragrance # 51226 | |
| | (and) | (Belle Aire Fragrances) | |
| | Musa Sapientum (Banana) Fruit | | |
| | Extract (and) Vitis Vinifera (Grape) | | |
| | Fruit Extract (and) Iris Florentina | | |
| | Root Extract (and) Hibiscus | | |
| | Abelmoschuus | | |
| 6 | Water (and) Butylene Glycol (and) | APT GC (Centerchem) | 0.01 |
| | Ahnfeltja Concinna Extract (and) | | |
| | Glycosaminoglycans | | |
| 6 | Acetyl Hexapeptide-3 | Argireline (Centerchem) | 0.01 |
| 6 | Water (and) Phospholipids (and) | Brookosome A, C, E, B5 | 0.10 |
| | Retinyl Palmitate (and) Tocopheryl | (Arch) | |
| | Acetate (and) Ascorbyl Palmitate | | |
| | (and) Pantothenic Acid (and) | | |
| 7 | Phenoxyethanol (and) Chlorphenesin | Germazide MPB | 1.00 |
| | (and) Glycerin (and) Methylparaben | (Collaborative) | |
| | (and) Benzoic Acid | | |

With reference to Table 14, the composition of Example 14 is prepared by combining the Seq. #1 materials in stainless steel, jacketed kettle, equip with propeller type agitation, homogenization and sweep, under adequate propeller type agitation. The Seq. # 2, Xanthan gum is then slowly sprinkled into the batch under adequate agitation. Top and bottom samples are checked for lumps. When the batch is homogeneous, it is heated to a target temperature of approximately 76-78 C. Under vigorous agitation, the Seq. #3,Z-Cote, is then slowly added, and the batch is mixed well. The zinc must be evenly and completely dispersed before proceeding. The Seq. #4 materials are then combined, under adequate agitation, in a jacketed side kettle, equipped with lightnin type agitation, and heated to 80-82 C. At proper temperatures, the Seq. #4 mixture is slowly added to the main batch, under homogenizer. The homogenization is maintained until the batch is stabilized and uniform, and is then switched to sweep and cooled to a target temperature of approximately 60 C. At 60° C. the Seq. # 5 material is added to the batch and the mixing speed is increased if necessary, to smooth the appearance of the batch. The batch is then cooled to approximately 40° C., and the Seq. # 6 materials are added, one at a time, mixing to uniformity in between additions. The Seq. #7 material is then added, and the batch is mixed to uniformity. The batch is then cooled to approximately 25° C. and top and bottom samples are compared to a standard.

### EXAMPLE 15

The composition of this example is preferably used as a skin firming cream, especially suitable for the eye area. The ingredients included in the composition are listed in Table 15:

TABLE 15

| Seq. | INCI Name | Trade Name | % W/W |
|---|---|---|---|
| 1 | Water (Aqua) | Water | 52.43 |
| 1 | Glycerin | Glycerin 99% | 3.00 |
| 1 | Disodium EDTA | Dissolvine Na2 (akzo) | 0.10 |
| 1 | Aloe Barbadensis Leaf Juice | Aloe barbadensis Gel (Lily of | 0.10 |
| | | the Desert/Texas Organics) | |
| 1 | PEG-12/SMDI Copolymer | Polyoperpolymer-2 (Barnet) | 2.00 |
| 1 | Methylparaben | Methylparaben | 0.25 |
| 2 | Sodium Acrylate Copolymer | Cosmedia SP (Cognis) | 0.30 |
| 3 | Magnesium Aluminum Silicate | Veegum HV (Vanderbilt) | 0.30 |
| 4 | Butter Butyrospermum Parkii (Shea Butter) | Shea Butter (Sederma) | 0.50 |
| | Fruit | | |
| 4 | Bisabolol | Bisabolol (Symrise) | 0.02 |
| 4 | Glyceryl Stearate (and) PEG-100 | Lipomulse 165 (Lipo) | 3.00 |
| | Stearate | | |
| 4 | PEG-4 Olivate | Olivem 700 (Presperse) | 2.00 |
| 4 | Sorbitan Olivate | Olivem 900 (Presperse) | 0.50 |
| 4 | Squalane | Squalane (Barnet) | 5.00 |
| 4 | Cetyl Lactate | Ceraphyl 28 (ISP) | 1.50 |
| 4 | Cetearyl Alcohol (and) Ceteareth-20 | Lipowax D (Lipo) | 3.50 |
| 4 | Vitis Vinifera (Grape) Seed Oil | Lipovol G/Grape Seed Oil | 1.00 |
| | | (Lipo) | |
| 4 | Dimethicone | Dow Corning 200/200 CTS | 1.50 |
| | | (Dow Corning) | |
| 4 | Propylparaben | Propylparaben | 0.10 |
| 4 | Hydrogenated Lecithin | Lecinol S-10 (Barnet) | 0.30 |
| 5 | PVP | PVP K-90 (ISP) | 0.80 |
| 6A | Nylon 12 | Orgasol 2002D Ex. Nat cos. | 3.00 |
| | | (Induchem/Lipo) | |
| 6B | Gossypium Herbaceum (Cotton), | Skin Fabric Silica (Resources | 1.50 |
| | Silica, Dimethicone | of Nature) | |
| 7 | Water, Phospholipids, Retinyl | Brookosome A, C, E, & B5* | 0.05 |
| | Palmitate, Tocopheryl Acetate, | (Arch) | |
| | Ascorbyl Palmitate, Pantothenic | | |
| | Acid | | |
| 7 | Water (and) Butylene Glycol (and) | APT-GC (Centerchem) | 2.00 |
| | Almfeltia Concinna Extract (and) | | |
| | Glycosaminoglycans | | |
| 7 | Acetyl Hexpeptide-3 | Argireline (Centerchem) | 5.00 |
| 7 | Hydrolyzed Rice Protein (and) | Regu-Age (Centerchem) | 5.00 |
| | Glycine Soja (Soybean) Protein (and | | |
| | Oxido Reductases | | |
| 7 | Water, Sodium Hyaluronate | Hyasol BT 1% (Centerchem) | 1.00 |
| 7 | Methyldihydrojasmonate | Hedione Maskant (Firmenich) | 0.25 |
| 7 | Phenoxethanol | Phenoxyethanol | 1.00 |
| 7 | Cyclomethicone | DC 345 Fluid (Dow Corning) | 3.00 |

With reference to Table 15, the composition of Example 15 is prepared by first combining the Seq. # 1 materials, under propeller agitation, in a stainless steel, jacketed main kettle, equipped with a variable speed homogenizer, sweep agitation, and propeller mixer, and heating to approximately 78-80 degrees centigrade. Under good homogenization, The seq. # 2 powder is slowly added by sprinkling the material slowly and evenly across the agitating water. Once free from lumps, it is cooled to 76-78 degrees centigrade. The Seq. #3 powder is then incorporated in the same manner, and the batch is mixed until uniform. Top and bottom samples are preferably checked at this point, before proceeding. In a side kettle, equipped with propeller type agitation, the Seq. # 4 materials are combined and heated to 80-82 C under adequate agitation. At proper temperatures, the Seq. # 4 materials are added to the main batch slowly while maintaining good agitation and temperature until the emulsion is well formed and stabilized. The batch is then cooled to a target temperature of 70 degrees centigrade. At 70 degrees, the Seq. # 6A material is added by slowly sprinking the powder on top of the batch, and with a sifting-type motion, being careful not to sift until the Nylon folds into the emulsion, since the Nylon forms clumps with itself if added too quickly. The seq. # 6B material is then added in the same manner. The Seq. #5 material is then added, adjusting the mixing if necessary. The batch is then cooled to a target temperature of 30-35 C, switching to sweep when necessary. At 30-35 degrees, the Seq. # 7 materials are added, one at a time, mixing well in between additions to uniformity. The batch is then cooled to approximately 25° C. and top and bottom samples are compared to a standard.

### EXAMPLE 16

The composition of this example is preferably used as a skin firming cream, especially suitable for the eye area. The ingredients included in the composition are listed in Table 16:

TABLE 16

| Seq. | INCI Name | Trade Name | % W/W |
|---|---|---|---|
| 1 | Water (Aqua) | Water | 34.17 |
| 1 | PEG-150 Distearate | Lipopeg 6000DS (Lipo) | 0.20 |
| 1 | Glycerin | Glycerin 99% | 4.00 |
| 1 | Disodium EDTA | Dissolvine Na2 (Akzo) | 0.10 |
| 1 | Aloe Barbadensis Leaf Juice | Aloe Barbadensis Gel (Lily of | 1.00 |
| | | the Desert) | |
| 1 | Methylparaben | Methylparaben | 0.25 |
| 1 | Propylparaben | Propylparaben | 0.10 |
| 2 | Chondrus Crispus (Carrageenan) | Viscarin SD 389 (FMC) | 0.55 |
| 3 | Butter Butyrospermum Parkii (Shea Butter) | Shea Butter (Sederma) | 0.50 |
| | Fruit | | |
| 3 | Bisabolol | Bisabolol (Lipo) | 0.02 |
| 3 | Glyceryl Stearate (and) PEG-100 | Lipomulse 165 (Lipo) | 3.00 |
| | Stearate | | |
| 3 | PEG-4 Olivate | Olivem 700 (Presperse) | 2.75 |
| 3 | Sorbitan Olivate | Olivem 900 (Presperse) | 0.50 |
| 3 | Squalane | Squalane (Barnet) | 8.00 |
| 3 | Cetyl Lactate | Ceraphyl 28 (ISP) | 3.00 |
| 3 | Cetearyl Alcohol (and) Ceteareth-20 | Lipowax D (Lipo) | 2.00 |
| 3 | Dimethicone | Dow Corning 200/200 CTS | 2.00 |
| | | (Dow Corning) | |
| 3 | Dipalmitoyl Hydroxyproline | Seppic DPHP (Seppic) | 1.00 |
| 3 | Hydrogenated Lecithin | Lecinol S-10 (Barnet) | 0.30 |
| 4 | Nylon-12 | Orgasol 2002 LTD Nat Cos. | 2.00 |
| | | (Lipo) | |
| 5 | Polyacrylamide (and) C 13-14 | Sepigel 305 (Seppic) | 0.50 |
| | Isoparaffin (and) Laureth-7 | | |
| 6 | Acetyl Hexapeptide-3 | Argireline (Centerchem) | 10.00 |
| 6 | Dimethylmethoxy Chromol | Lipochroman 6 (Centerchem) | 0.02 |
| 6 | Water, Hydrolyzed Wheat Protein, | Aldenine (Centerchem) | 2.00 |
| | Hydrolyzed Soy Protein, Xanthan | | |
| | Gum, Tripeptide-1, | | |
| | Phenoxyethanol, | | |
| | Methylparaben, Propylparaben, | | |
| | Butylparaben, Ethylparaben | | |
| 6 | Camellia Sinesis Leaf Extract | White Tea (Barnet) | 0.02 |
| 6 | Butylene Glycol, Water, Camellia | Actyphyte of Japanese Green | 0.02 |
| | Oleifera Leaf Extract | Tea BG50 | |
| 6 | Saccharide Isomerate | Pentavitin (Centerchem) | 2.00 |
| 6 | Water (and) Butylene Glycol (and) | APT-GC (Centerchem) | 2.00 |
| | Ahnfeltia Concinna Extract (and) | | |
| | Glycosaminoglycans | | |
| 6 | Hydrolyzed Rice Protein (and) | Regu-age (Centerchem) | 5.00 |
| | Glycine Soja (Soybean) Protein (and) | | |
| | Oxido Reductases | | |
| 6 | Ceramjde 3 (and) Ceramide 3 (and) | Skn Influx (Centerchem) | 3.00 |
| | Ceramide 6 (and) Ceramide I (and) | (Cholesterol-Free Version) | |
| | Phytosphingosine (and) Sodium | | |
| | Lauroyl Lactylate (and) Carbomer | | |
| | (and) Xanthan Gum (and) | | |
| | Methylparaben (and) Propylparaben | | |
| 6 | Water (Aqua), Glycerin, Steareth-20 | Haloxyl (Sederma) | 3.00 |
| | N-Hydroxysuccinimide-Chrysin | | |
| | Palmitoyl Oligopeptide, Palmitoyl | | |
| | Tetrapeptide-3 | | |
| 6 | PEG-6 Isostearate (and) Hesperetin | Flavagrum PEG (Coletica) | 3.00 |
| | Laurate | | |
| 6 | Sodium Hyaluronate | Hyasol BT (Centerchem) | 2.00 |
| 6 | Cucumis Sativis (Cucumber) Fruit | Actiphyte of Cucumber GL5O | 0.50 |
| | Extract | (Active Organics) | |
| 6 | Glycyrrhiza Glabra (Licorice) | Actiphyte of Licorice GL5O | 0.50 |
| | Extract | (Active Organics) | |
| 6 | Phenoxyethanol | Emmeressence 1160 Rose | 1.00 |
| | | (Cognis) | |

With reference to Table 16, the composition of Example 16 is prepared by first combining the Seq. # 1 materials in stainless steel, jacketed main kettle, equipped with sweep agitation and propeller mixer, under propeller mixing, and heating to 78-80 degrees centigrade. Under ample propeller agitation, the seq. # 2 Carrageenan powder is slowly added by sprinkling slowly and evenly across the agitating water, being careful not to aerate the batch, since carrageenan has suspension properties. After checking that the batch is free from lumps on top and bottom samples, the batch is cooled to 76-78 degrees centigrade. Alternatively, the Seq. #2 Carrageenan can be added prior to heating. In a jacketed, stainless steel side kettle with adequate propeller agitation, the seq. # 3 materials are combined and the batch is heated to a target temperature of 80-82 degrees centigrade. At proper temperatures, the Seq. # 3 batch is slowly added to the main batch, while maintaining good agitation and temperature, until an emulsion is well formed and stabilized. The batch is then cooled to 70 degrees centigrade. At 70 degrees, the Seq. # 4 Nylon 12 powder is added by slowly sprinkling the powder on top of the batch, with a sifting-type motion, being careful not to sift until the Nylon folds into an emulsion, since the Nylon will form clumps with itself if added too quickly. The batch is then cooled to 50 degrees centigrade. At 50 degrees centigrade, the Seq. #5 Sepigel is added, being sure that the drum is well mixed before adding since this material separates in storage. At this point in may be necessary to increase mixing speed until the batch is smooth and uniform; then resume prior speed or sweep agitation. The batch is then cooled to 35 C, and at 35 degrees, the Seq. # 6 materials are added to the batch, one at a time, mixing until evenly distributed. The batch is then cooled to approximately 25° C. and top and bottom samples are compared to a standard.

### EXAMPLE 17

The composition of this example is preferably used as an eye serum or semi-viscous gel. The ingredients included in the composition are listed in Table 17:

TABLE 17

| Seq. | INCI Name | Trade Name | % W/W |
|---|---|---|---|
| 1 | Water (Aqua) | Deionized Water | 52.979 |
| 1 | Glycerin | Glycerin 99% | 5.000 |
| 1 | Panthenol | Panthenol | 0.100 |
| 1 | Aloe Barbadensis Leaf Juice | Aloe Vera Juice (Lily of the | 0.050 |
| | | Desert) | |
| 1 | Disodium EDTA | Dissolvine Na2 | 0.100 |
| 2 | Hydroxyethylcellulose | Natrosol 250 HHR | 1.000 |
| 3 | (Proposed INCI Name) Water, Glycerin | Leuphasyl (Centerchem) | 5.000 |
| | Caprylyl Glyceride, Pentapeptide-3 | | |
| 3 | Water, Hexapeptide-10 | Serilesesine (Centerchem) | 2.000 |
| 3 | Palmitoyl Tripeptide-3, Glycerin | Syn-Coll (Centerchem) | 2.000 |
| 3 | Acetyl Hexapeptide-3 | Argireline (Centerchem) | 10.000 |
| 3 | Zea (Corn) Mays Extract | Deliner (Coletica) | 2.000 |
| 3 | Glycyrrhiza Glabra (Licorice) Extract | Licorice extract (Carrubba) | 1.000 |
| | | #C6949/WS | |
| 3 | Hydrolyzed Soy Protein | Phytokine (Engeihard/Coletica) | 1.000 |
| 3 | Dimethylmethoxy Chromanol | Lipochroman 6 (Centerchem) | 0.020 |
| 3 | Camellia Sinensis (Green) Tea | Japanese Green Tea GL: 50 | 1.000 |
| | | (Active Organics) | |
| 3 | Camellia Sinensis (White) Leaf Tea | White Tea Extract GL: 50 (Active | 1.000 |
| | | Organics) | |
| 3 | Water, Hydrolyzed Wheat Protein, Hydrolyzed | Aldenine (Centerchem) | 0.001 |
| | Soy Protein, Xanthan Gum, Tripeptide-1, | | |
| | Phenoxyethanol, Methylparaben, | | |
| | Propylparaben, Butylparaben, Ethylparaben | | |
| 3 | Water, Butylene Glycol, Cola Nitida | Quench-T (Centerchern) | 3.000 |
| | Seed Extract, Paullina Cupana Seed | | |
| | Extract, Ilex Paraguariensis | | |
| 3 | Water, Butylene Glycol, Ahnfeltia | APT-GC (Centerchem) | 2.000 |
| | Concinna Extract Glycosaminoglycans | | |
| 3 | Water, Sodium Hyaluronate | Hyasol 1% BT (Centerchem) | 2.000 |
| 3 | Water, Alcohol, Foeniculum Vulgare | Sediplant Richter (TAOS) | 2.000 |
| | (Fennel) Fruit Extract, Humulus | | |
| | Lupulus (Hops) Extract, Melissa | | |
| | Officinalis Leaf Extract, Viscum Album | | |
| | (Mistletoe) Leaf Extract, Chamomilla | | |
| | Reticutita (Matricaria) Flower Extract, | | |
| | Achillea Millefolium Extract Urea, | | |
| | Allantoin | | |
| 3 | Water, Glycerin, Steareth-20, N- | Haloxyl (SedermalCroda) | 3.000 |
| | Hydroxysuccinimide, Chrysin, | | |
| | Palmitoyl Oligopeptide, Palmitoyl | | |
| | Peptide-3 | | |
| 3 | Acetyl Tetrapeptide-5 | Eyeseryl (Centerchem) | 3.000 |
| 3 | Phenoxyethanol, Caprylyl Glycol, | Jeecide CAP-S (Jeen) | 0.750 |
| | Potassium Sorbate, Hexelene Glycol | | |

With reference to Table 17, the composition of Example 17 is prepared by first combining the Seq. # 1 materials in stainless steel, jacketed main kettle, equipped with a variable speed propeller mixer and sweep agitation, under adequate mixing so that the entire tank is circulating. The batch is heated to about 80 C to eliminate the presence of gram positive bacteria which may be in the water. Either prior to or during the heating process, the Seq. #2 material is added very slowly under high-energy propeller. When uniformly incorporated, top and bottom samples are preferably checked for lumps, being careful not to over aerate the batch. Once the batch is clear and free of lumps, it is cooled to 35 C and switched to sweep as the batch thickens. At 35 C the Seq. #3 materials are added, one at a time, mixing to uniformity in between additions. The batch should remain clear with each addition. Top and bottom samples may be compared to a standard at this point.

### EXAMPLE 18

The composition of this example is preferably used a serum for firming the skin and treating wrinkles. The ingredients included in the composition are listed in Table 18:

TABLE 18

| Seq. | INCI Name | Trade Name | % W/W |
|---|---|---|---|
| 1 | Water (Aqua) | Deionized Water | 63.68 |
| 1 | Methylparaben | Methylparaben | 0.25 |
| 1 | Glycerin | Glycerin 99% | 5.00 |
| 1 | Panthenol | Panthenol (Roche) | 0.10 |
| 1 | Aloe Barbadensis Leaf Juice | Aloe Vera Juice (Lily of the Desert) | 0.05 |
| 1 | Disodium EDTA | Dissolvine NA2 (Akzo) | 0.10 |
| 2 | Hydroxyethylcellulose | Natrosol 250 HHR | 1.25 |
| | | (Hercules/Aqualon) | |
| 3 | (Proposed INCI Name) Water, | Leuphasyl (Centerchem) | 5.00 |
| | Glycerin | | |
| | Caprylyl Glyceride, Pentapeptide-3 | | |
| 3 | Water, Hexapeptide-10 | Serilesesine (Centerchem) | 2.00 |
| 3 | Palmitoyl Tripeptide-3, Glycerin | Syn-Coll (Centerchem) | 2.00 |
| 3 | Acetyl Hexapeptide-3 | Argireline (Centerchem) | 10.00 |
| 3 | Water Glycerin Ahnfeltia Concinna | APT-GC (Centerchem) | 2.00 |
| | Extract, Glycosaminoglycans | | |
| 3 | Zea (Corn) Mays Extract | Deliner (Coletica) | 2.00 |
| 3 | Glycyrrhiza Glabra (Licorice) Extract | Licorice extract (Carrubba) | 0.10 |
| 3 | Hydrolyzed Soy Protein | Phytokine (Coletica) | 1.00 |
| 3 | Dimethylmethoxy Chromanol | Lipochroman 6 (Centerchem) | 0.02 |
| 3 | Water, Hydrolyzed Wheat Protein, | Aldenine (Centerchem) | 2.00 |
| | Hydrolyzed Soy Protein, Xanthan | | |
| | Gum, Tripeptide-1, Phenoxyethanol, | | |
| | Methyparaben, Propylparaben, | | |
| | Butylparaben, Ethylparaben | | |
| 3 | Camellia Oleifera (Japanese Green) | Actiphyte of Japanese Green Tea GL | 0.10 |
| | Tea Extract | 50 (Active Organics) | |
| 3 | Camellia Sinesis (White) Tea Extract | White Tea Extract (Barnet) | 0.10 |
| 3 | Sodium Hyaluronate | Hysol 1% BT (Centerchem) | 1.00 |
| 4 | Cucumis Melo (melon) Extract | Liquid Melon Crodarome (Croda) | 0.25 |
| 4 | PPG-Ceteth-20 | Procetyl AWS (Croda) | 1.00 |
| 4 | Phenoxyethanol | Phenoxyethanol | 1.00 |

With reference to Table 18, the composition of Example 18 is prepared by first combining the Seq. # 1 materials in stainless steel, jacketed main kettle, equipped with a variable speed propeller mixer and sweep agitation, under adequate mixing so that the entire tank is circulating. The batch is heated to about 80 C to eliminate the presence of gram positive bacteria which may be in the water. Either prior to or during the heating process, the Seq. #2 material is added very slowly under high-energy propeller. When uniformly incorporated, top and bottom samples are preferably checked for lumps, being careful not to over aerate the batch. Once the batch is clear and free of lumps, it is cooled to 35 C and switched to sweep as the batch thickens. At 35 C the Seq. #3 materials are added, one at a time, mixing to uniformity in between additions. The Seq. #4 materials are premixed until clear and uniform, and slowly trickled near the energy source while adding. The batch may cloud slightly with the addition, and mix to clarity. Top and bottom samples may be compared to a standard at this point.

### EXAMPLE 19

The composition of this example is preferably used a mask for firming the skin and treating wrinkles. The ingredients included in the composition are listed in Table 19:

TABLE 19

| Seq. | INCI Name | Trade Name | % W/W |
|---|---|---|---|
| 1 | Water (Aqua) | Deionized Water | 67.48 |
| 1 | Methylparaben | Methylparaben | 0.25 |
| 1 | Glycerin | Glycerin 99% | 5.00 |
| 1 | Panthenol | Panthenol (Roche) | 0.10 |
| 1 | Aloe Barbadensis Leaf Juice | Aloe Vera Juice (Lily of the | 0.05 |
| | | Desert) | |
| 1 | Disodium EDTA | Dissolvine Na2 (Akzo) | 0.10 |
| 2 | Xanthan Gum | Keltrol CG (Keltrol) | 0.33 |
| 3 | Algae Extract and Pullulan | Pepha-Tight (Centerchem) | 3.00 |
| 4 | Palmitoyl Tripeptide-3, Glycerin | Syn-Coll (Centerchem) | 2.00 |
| 4 | Acetyl Hexapeptide-3 | Argireline (Centerchem) | 10.00 |
| 4 | Zea (Corn) Mays Extract | Deliner (Coletica) | 2.00 |
| 5 | Water, Ahnfeltia Concinna Extract | APT-GC (Centerchem) | 2.00 |
| | Glycosaminoglycans | | |
| 5 | Dimethylmethoxy Chromanol | Lipochroman 6 (Centerchem) | 0.02 |
| 5 | Water, Hydrolyzed Wheat Protein, | Aldenine (Centerchem) | 2.00 |
| | Hydrolyzed Soy Protein, Xanthan | | |
| | Gum, Tripeptide-1, Phenoxyenthanol, | | |
| | Methylparaben, Propylparaben, | | |
| | Butylparaben, Ethylparaben | | |
| 5 | Camellia Oleifera (Japanese Green) | Actyphyte of Japanese Green Tea | 0.10 |
| | Tea Extract, Water, Glycerin | GL 50 (Active Organics) | |
| 5 | Camellia Sinensis (White) Tea Extract | White Tea Extract (Barnet) | 0.10 |
| 5 | Ascorbic-Acid-2 Glucoside) | Blucoside AA2-G (DKSH) | 0.02 |
| 5 | Cucumis Melo (Melon) Extract | Liquid Melon Crodarome | 0.25 |
| | | (Croda) | |
| 5 | Sorbitol, Water, Algae Extract | Lana Blue (Atrium) (TAOS) | 3.00 |
| 5 | Mica & Titanium Dioxide (CI 77891) | Interval Red (Kobo) | 0.20 |
| 5 | Water, Sodium Hyaluronate | Hyasol BT 1% (Centerchem) | 1.00 |
| 6 | Phenoxyethanol | Phenoxyethanol | 1.00 |

With reference to Table 19, the composition of Example 19 is prepared by first combining the Seq. # 1 materials, one at a time under adequate agitation, in stainless steel main kettle, equipped with a variable speed propeller-type agitation. The batch is heated to about 80 C to eliminate the presence of gram positive bacteria, and dissolve the parabens. The batch is then cooled to 35-40 degrees Centigrade. During the beginning of the cooling process, the Seq. #2 material is added under adequate propeller energy, until the batch is uniform. Top and bottom samples are preferably checked before continuing, and the batch is continued to cool. At 35-40 degrees, the Seqs. #3, 4, 5, 6, and 7 materials are added, one at a time, mixing to uniformity with each addition. Top and bottom samples may be compared to a standard at this point.

### EXAMPLE 20

The composition of this example is preferably used a wrinkle treatment lotion, preferably for the lips. The ingredients included in the composition are listed in Table 20:

TABLE 20

| | | | |
|---|---|---|---|
| 1 | Water (Aqua) | Deionized Water | 51.1 |
| 1 | Glycerin | Glycerin 99% | 8.50 |
| 1 | Disodium EDTA | Dissolvine Na2 (Akzo) | 0.10 |
| 1 | Methylparaben | Methylparaben | 0.25 |
| 1 | PEG-75 | Pluracol E4000 | 0.40 |
| | | Pastille (BASF) | |
| 1 | PEG-40 Stearate | Lipopeg-39S (Lipo) | 0.40 |
| 2 | Cetearyl Alcohol (and) Polysorbate 60 | Procol P (Protameen) | 4.50 |
| | (and) PEG-150 Distearate (and) | | |
| | Steareth-20 | | |
| 2 | Tridecyl Stearate (and) Polysorbate 60 | Lipovol MOS-70 (Lipo) | 4.15 |
| | (and) PEG-150 Distearate (and) | | |
| | Steareth-20 | | |
| 2 | Glyceryl Stearate, PEG-100 Stearate | Lipomulse-165 (Lipo) | 2.50 |
| 2 | Polyethylene | Performalene 400 | 0.40 |
| | | Polyethylene (New Phase) | |
| 2 | Ethylhexyl Palmitate | Ceraphyl 368 ISP | 0.85 |
| 2 | Dimethicone | Dow Corning (Dow Corning) | 0.10 |
| | | 200/200 cts | |
| 2 | Sorbitan Stearate | Protachem SMS (Protameen) | 0.35 |
| 2 | Propylparaben | Proplyparaben | 0.10 |
| 3 | Ethyhexyl Palmitate, Tribehenin, | Maxi-Lip (Croda/Sederma) | 3.00 |
| | Soribtan Isostearate, Palmitoyl | | |
| | Oligopeptide | | |
| 4 | Aluminum Starch Octenysuccinate | Skin Flo (Midwest Grain) | 3.00 |
| 5 | Water | Water | 1.00 |
| 5 | Phenoxyethanol | Phenoxyethanol | 1.00 |
| 5 | Water (and) Phospholipid (and) | *Brookosome | 1.00 |
| | Tocopheryl Acetate (and) Retinyl | ACEB5 (Arch) | |
| | Palmitate (and) Ascorbyl Palmitate | | |
| 5 | Benzalkonium Choloride | Benzalkonium Choloride | 0.05 |
| 6 | Water, Hydrolyzed Wheat Protein, | Aldenine (Centerchem) | 0.02 |
| | Hydrolyzed Soy Protein, Xanthan | | |
| | Gum, Tripeptide-1, Phenoxyethanol, | | |
| | Methyparaben, Propylparaben, | | |
| | Butylparaben, Ethylparaben | | |
| 6 | Dimethylmethoxy Chromanol | Lipochroman 6 (Centerchem) | 0.01 |
| 6 | Camellia Sinesis Leaf Extract | White Tea (Barnet) | 0.02 |
| 6 | Butylene Glycol, Water, Camellia | Actyphyte of Japanese Green Tea | 0.02 |
| | Oleifera Leaf Extract | BG 50 (Active Organics) | |
| 6 | Acetyl Hexapeptide-3 | Argireline (Centerchem) | 10.00 |
| 6 | Water, Butylene Glycol | APT-GC (Centerchem) | 2.00 |
| | Glycosaminoglycans Ahnfeltia | | |
| | Concinna Extract | | |
| 6 | Water, Sodium, Hyaluronate | Hyasol BT 1% | 5.00 |
| | | (Centerchem) | |

With reference to Table 20, the composition of Example 20 is prepared by first combining the Seq. # 1 materials, in a stainless steel, jacketed kettle equipped with lightnin type agitation and a sweep mixer, and heating to 76-78 degrees centigrade under adequate lightnin' agitation. In an auxiliary, stainless steel kettle equip with a lightnin' type mixer, the Seq. #2 materials are combined and heated to 80-82 degrees centigrade. At proper temperatures, the Seq. # 2 batch is slowly added to the Seq. # 1 main kettle, under adequate agitation, maintaining the temperature and agitation until an emulsion is well formed. The batch is then cooled to 42 C and the setting is switched to sweep as the batch thickens. At 42 C the premixed Seq. # 3 materials are added to the batch. The Seq. #4 materials are then added to the batch. The batch is then cooled to 25 C and samples may be compared to a standard.

Though the term “batch” is used in the above examples, it is to be understood that the compositions may be formed in batch or continuous processes.

In closing, it is to be understood that the exemplary embodiments described herein are illustrative of the principles of the present invention. Other modifications that may be employed are within the scope of the invention. Thus, by way of example, but not of limitation, alternative configurations may be utilized in accordance with the teachings herein. Accordingly, the description is illustrative and not meant to be a limitation thereof.

1. A skin care composition, comprising:
a muscular contraction inhibiting agent comprising an effective amount of acetyl hexapeptide Argireline®, whereby said acetyl hexapeptide specifically targets the repeated, biomechanical, muscular contractions of facial expressions by functioning to reduce the intensity of these muscle contractions, and further reduces the release of catecholamines and limits the production of SNARE complexes,
an exfoliation component comprising an effective amount of Ahnfeltia Concinna extract, whereby said Ahnfeltia Concinna extract increases cellular turnover and density of collagen bundles of the upper dermis of the skin;
said composition being formulated in a physiologically acceptable medium, whereby components a) and b) work in concert to reduce wrinkles and improve overall skin appearance.

2. The skin care composition of claim 1, further comprising melon, banana, and grape extract.

3. The skin care composition of claim 2, further comprising Iris Florentina Root Extract and Hibiscus Abelmoschuus.

4. The skin care composition of claim 1, further comprising at least one additional peptide component selected from the group comprising a tripeptide, a tetrapeptide, a pentapeptide, an oligopeptide, hexapeptide Serilesine®, and derivatives thereof.

5. The skin care composition of claim 1, further comprising willow bark extract.

6. The skin care composition of claim 1, further comprising corn extract.

7. The skin care composition of claim 1, further comprising hydrolyzed rice protein, Soybean Protein, and Oxido Reductases.

8. The skin care composition of claim 1, further comprising dypalmitoyl hydroxyproline.

9. The composition of claim 1, further comprising imparata cylindrical extract.

10. The skin care composition of claim 1, further comprising magnesium ascorbyl phosphate, tocopherol, lecithin, dypalmitoyl hydroxyproline, melon, banana, and grape fruit extract, iris root extract, and hibiscus abelmoschuus.

11. The skin care composition of claim 1, further comprising dypalmitoyl hydroxyproline, Imperata Cylindrica Root Extract, Magnesium Ascorbyl Phosphate, Tocoperol, and melon extract.

12. The skin care composition of claim 1, further comprising willow bark extract, citrus aurantium amara extract, magnesium ascorbyl phosphate, tocopherol, and imparata cylindrical extract.

13. The composition of claim 1, further comprising olive oil, melon extract, and grape seed extract.

14. The composition of claim 1, further comprising a skin benefiting ingredient selected from the group comprising: Dipalmitoyl Hydroxyproline, Melon Extract, Banana Fruit Extract, Vitis Vinifera (Grape) Fruit Extract, Iris Florentina Root Extract, Hibiscus Abelmoschuus, Magnesium Ascorbyl Phosphate, Imperata Cylindrica Root Extract, willow bark extract, Citrus Aurantium Amara (Bitter Orange) Flower Extract, corn extract, Hypnea Musciformis Extract, Gellidiela Acerosa Extract, Sargassum Filipendula Extract, Humulus Lupulus (Hops) Extract, Rosmarinus Officinalis, Equisetum Arvense Extract, Pinus Sylvestris Cone Extract, Citrus Medica Limonum (Lemon) Fruit Extract, Vitis Vinifera (Grape) Leaf Extract, Glycine Soya (Soybean) Germ Extract, Triticum Vulgare (Wheat) Germ Extract, Vitis Vinifera (Grape) Seed Extract, Ceramide 3, Ceramide 6, Ceramide 1, Cholesterol, Phytosphingosine, Hexapeptide-10, Palmitoyl Tripeptide-3/tetrapeptide-3, pentapeptide-3, tripeptied-1, palmitoyl oligopeptide, palmitoyl peptide-3, actyl tetrapeptide-5, citrus grandis (grapefruit) Peel extract, Vitamins A, C, E, and B5, Hydrolyzed Rice Protein, Glycine Soja (Soybean) Protein, Oxido Reductases, Vitis Vinifera (Grape) Seed Oil, Hydrolyzed Wheat Protein, Hydrolyzed Soy Protein, Tripeptide-1, green tea, white tea, cucumber fruit extract, licorice extract, Cola Nitida Seed Extract, Paullina Cupana Seed Extract, Ilex Paraguariensis, Foeniculum Vulgare (Fennel) Fruit Extract, Humulus Lupulus (Hops) Extract, Melissa Officinalis Leaf Extract, Viscum Album (Mistletoe) Leaf Extract, Chamomilla Reticutita (Matricaria) Flower Extract, Achillea Millefolium Extract Urea, Allantoin, oat beta glucan, and combinations thereof.
